(19) United States
(12) Patent Application Publication
(10) Pub. No.: US 2026/0283699 A1
(43) Pub. Date: Sep. 24, 2026

(54) SYSTEM AND METHOD FOR POSITIONING A MEMBER RELATIVE TO A SUBJECT

(71) Applicant: Medtronic, Inc., Minneapolis, MN (US)

(72) Inventors: Elliot C. Schmidt, Minneapolis, MN (US); Varun A. Bhatia, Minneapolis, MN (US); Ryan P. Lahm, Lino Lakes, MN (US); Matthew J. Hoffman, Saint Paul, MN (US); Linnea R. Lentz, Stacy, MN (US); Tarek D. Haddad, Minneapolis, MN (US); Brett D. Jackson, Shoreview, MN (US)

(21) Appl. No.: 19/474,681

(22) PCT Filed: Apr. 10, 2024
(86) PCT No.: PCT/US2024/023918

§ 371 Date: Oct. 10, 2025

Related U.S. Application Data
(60) Provisional application No. 63/459,040, filed on Apr. 13, 2023.

(51) Int. Cl. A61B 34/20; A61B 34/00; A61B 34/10; A61B 90/00; A61N 1/05; A61N 1/362

(52) U.S. Cl. CPC A61B 34/20; A61N 1/0587; A61N 1/362; A61B 2034/105; A61B 2034/2051; A61B 2034/2065; A61B 2034/256; A61B 2090/378

(57) ABSTRACT

Disclosed is a system to display a representation of an instrument. The representation of the instrument may be displayed relative to a representation of a patient. The representation of the patient may be fit in real-time to represent poses or one or more portions of the patient relative to one another and/or to the instrument.

### CROSS-REFERENCE TO RELATED APPLICATION(S)

The present application claims the benefit of priority to 63/459,040, filed Apr. 13, 2023. This application includes subject matter that is similar to that disclosed in U.S. Pat. App. No. 63/459,112 (Attorney Docket No. A0009842US01/5074D-000094-US-PS1). The entire disclosure of each of the above applications is incorporated herein by reference.

### TECHNICAL FIELD

The present disclosure relates to positioning a member relative to a subject, and particularly to determining a pose of the member relative to the subject.

### BACKGROUND

This section provides background information related to the present disclosure which is not necessarily prior art.

An imaging system can be used to image various portions of a subject. The subject can include a patient, such as a human patient. The portions selected to be imaged can be internal portions that are covered by skin or other tissue. A location of imaged portions of the subject may be selected to be known within the imaging system. The locations can be defined or established relative to instruments placed in the subject (e.g., a location of a heart wall relative to a catheter) or a location of the imaged portion relative to the instrument acquiring the image data.

With assistance of an imaging modality an implant may be placed. The implant may be placed relative to a heart of a subject. Exemplary systems include the Medtronic Subcutaneous Lead System model 6996SQ that may be implanted subcutaneously with the use of the Medtronic Tunneling Tool model 6996T.

### SUMMARY

This section provides a general summary of the disclosure and is not a comprehensive disclosure of its full scope or all of its features.

The subject disclosure relates to illustrating a position, including a pose of an instrument (e.g., tunneling tool and/or lead) relative to a patient. The patient may be a human or non-human patient. Further, the subject may be a non-living object where the location of an instrument within the subject is selected to be tracked.

A representation of the patient may be fit or morphed to illustrate or represent a size of the patient. Therefore, relative portions of the patient, such as relative portions of the anatomy, are determined or known relative to one another based upon the patient that is the current patient during a procedure. An avatar or indicia of the patient may be generated or altered to a specific patient substantially in real time during a procedure.

An instrument (e.g., tunneling tool and/or lead) can be tracked relative to the patient. A representation of the instrument may be superimposed on the avatar of the patient. The avatar of the patient may provide a visual illustration of various portions of the anatomy of the patient without requiring image data being acquired of the patient. The display being viewed by the user may be generated without requiring image data of the patient, such as fluoroscopy image data.

The tracked instrument may be tracked with an appropriate tracking system. As discussed herein, the determined pose of the instrument or at least a portion of the instrument may be determined and illustrated relative to an image of the subject and/or the avatar. The instrument may include an introducer (e.g., for introducing a lead for stimulating a heart) or other appropriate instrument.

Further areas of applicability will become apparent from the description provided herein. The description and specific examples in this summary are intended for purposes of illustration only and are not intended to limit the scope of the present disclosure.

The present technology is illustrated, for example, according to various aspects described below, including with references to FIGS. 1-18B. Various examples of aspects of the present technology are described as numbered clauses (1, 2, 3, etc.) for convenience. These are provided as examples and do not limit the subject technology.

1. A system to track a device relative to a subject, comprising:
a display device configured to display an image that includes a representation of one or more internal portions of the subject;
an instrument configured to be moved relative to the heart of the subject, wherein the instrument includes a distal portion configured to be percutaneously positioned in the subject and a handle configured to move the distal portion;
a tracking system;
a tracking device configured to be tracked by the tracking system, wherein the tracking device is associated with the instrument;
a processing unit configured to execute instructions to:
   generate the image that includes the representation of the one or more internal portions of the subject;
   register the image to the subject; and
   generate a graphical representation of a pose of the instrument relative to the generated image; and
   a display device configured to display both the image and the graphical representation;
wherein the display is operable to illustrate at least a distance of the instrument relative to the heart of the subject based at least on the displayed image and the graphical representation.

2. The system of clause 1, further comprising:
an imaging system configured to generate image data of the one or more internal portions of the subject;
wherein processing unit is configured to generate the image with the image data.

3. The system of clause 2, further comprising:
an imaging system tracker, wherein the imaging system tracker is configured to be tracked by the tracking system to determine a pose of the imaging system relative to the subject;
wherein the imaging system is an ultrasound imaging system configured to generate the image data of the subject in a plane relative to an ultrasound transducer.

4. The system of clause 3, wherein the generated image is an image based on the ultrasound image data;
wherein the generated image is registered to the subject based at least on the imaging system tracker pose.

5. The system of any one of clauses 2-4, wherein the processing unit is configured to execute further instructions to:
recall a heart atlas;
morph the heart atlas based on the image data of the subject to generate a morphed atlas; and
display the morphed atlas as the image and the generated graphical representation relative to the morphed atlas.

6. The system of clause 5, wherein the atlas is registered to a generated image of the heart at least by an identification of a landmark in both the atlas and the generated image of the heart.

7. The system of any one of clauses 1-6, further comprising:
an implant configured to be implanted within the subject near the heart, wherein the implant is configured to include an electrode configured to deliver energy to the heart of the subject;
an implant tracking device associated with the implant;
wherein the implant tracking device is configured to be tracked with the tracking system to determine a pose of the implant.

8. The system of clause 7, wherein the tracking system includes an electrical impedance tracking system;
wherein the implant tracking device is the electrode of the implant;
wherein the processing unit is configured to execute further instructions to determine at least an orientation of the electrode.

9. The system of clause 8, wherein the determining orientation of the electrode includes at least a determination of a direction relative to a center line of the subject of an opening of a selected shape defined by the electrode.

10. The system of clause 9, wherein the electrode of the implant includes at least a first electrode and a second electrode;
wherein the first electrode includes a coil of electrically conductive material wrapped around the implant, wherein the first electrode is configured to achieve a “C” shape within the subject;
wherein the second electrode includes a ring electrode.

11. The system of any one of clauses 1-10, wherein the instrument is configured to be at least one of positioned between a sternum and the heart of the subject, subcutaneously, intercostally, intrapleurally, pericardially, epicardially, into a posterior mediastinum, or combinations thereof.

12. A method to track a device relative to a heart of a subject, comprising:
providing an instrument including (1) a distal portion configured to be percutaneously positioned and moved relative to the subject and (2) a handle configured to be manipulated to move the distal portion;
tracking a tracking device associated with the provided instrument;
operating a processing unit to execute instructions to:
generate an image to represent at least a portion of the heart of the subject;
register the image to the subject;
determine a pose of the provided instrument based on the tracking of the tracking device; and
generate a graphical representation of the instrument relative to the generated image based on the determined pose of the provided instrument; and
displaying with a display device both the generated image and the graphical representation;
wherein the display is operable to illustrate at least a distance of the instrument relative to the heart of the subject based at least on the displayed image and the graphical representation.

13. The method of clause 12, further comprising:
acquiring image data of the subject with an imaging system;
operating the processing unit to generate the image with the acquired image data.

14. The method of clause 13, further comprising:
tracking the imaging system with an imaging system tracker to determine a pose of the imaging system relative to the subject;
providing the imaging system as an ultrasound imaging system;
wherein operating the processing unit to register the image to the subject is based at least on the imaging system determined pose.

15. The method of any one of clauses 12-14, wherein operating the processing unit further includes:
recalling a heart atlas;
morphing the heart atlas based on the image data of the subject to generate a morphed atlas; and
displaying the morphed atlas as the image and the generated graphical representation relative to the morphed atlas.

16. The method of any one of clauses 12-15, further comprising:
providing an implant configured to be implanted within the subject near the heart,
providing the implant to include an electrode to deliver energy to the heart of the subject;
tracking the implant with an implant tracking device associated with the implant; and
determining a pose of the implant based on tracking the implant.

17. The method of clause 16, further comprising:
determining an orientation of the electrode including at least a determination of an orientation of non-symmetrical lead elements relative to a central axis of the implant.

18. The method of clause 16 or 17, wherein providing the implant to include the electrode includes providing the implant to include at least a first electrode and a second electrode; and
providing the first electrode to include a coil of electrically conductive material wrapped around the implant configured to achieve a “C” shape within the subject.

19. The method of any one of clauses 12-18, further comprising: providing an ultrasound imaging system and an x-ray imaging system.

20. A system to track a device relative to a heart of a subject, comprising:
a display device configured to display an image that includes a representation of the subject;
an instrument configured to be moved relative to the heart of the subject, wherein the instrument includes (1) a tunneling portion configured to be positioned transcutaneous between a sternum and the heart of the subject and (2) a handle configured to move the tunneling portion;
a lead configured to be positioned with a tunnel formed by the tunneling portion within the subject;
a tracking system;
an instrument tracking device configured to be tracked by the tracking system, wherein the instrument tracking device is associated with the instrument and tracked by the tracking system to determine an instrument pose;
a lead tracking portion configured to be tracked by the tracking system, wherein the lead tracking device is associated with the lead and tracked by the tracking system to determine a lead pose;
a processing unit configured to execute instructions to:
   generate the image that represents at least a portion of the heart of the subject based at least on a determined pose of the subject;
   register the image to the subject; and
      generate a graphical representation of the determined pose of at least one of the instrument or the lead relative to the generated image; and
a display device configured to display both the image and the graphical representation to illustrate the determined pose in real time relative to the heart of the subject, wherein the real time display illustrates at least a distance of the instrument relative to the heart of the subject based at least on the displayed image and the graphical representation.

### BRIEF DESCRIPTION OF THE DRAWINGS

The drawings described herein are for illustrative purposes only of selected embodiments, not all possible implementations, and are not intended to limit the scope of the present disclosure.

FIG. 1 is an environmental view of an imaging and navigation system;

FIG. 2 is a perspective view of an instrument operable to be used during a procedure, according to various embodiments;

FIG. 3 is a screenshot of a representation of an instrument at a tracked pose relative to an avatar, according to various embodiments;

FIG. 4 is a screenshot of a portion of the subject and a graphical representation of an instrument, according to various embodiments;

FIG. 5A is a screenshot of a generated image of the subject and a graphical representation of an instrument, according to various embodiments;

FIG. 5B is an alternative view of a subject and a graphical representation of an instrument, according to various embodiments;

FIG. 6 is a diagram of an electrical impedance tracking system, according to various embodiments;

FIG. 7 is a representation of an implant, according to various embodiments;

FIG. 8 is a detail view of the subject and of an imaging system in a first position, according to various embodiments;

FIG. 9 is a schematic view of a position of the imaging system housing relative to a portion of the subject, according to various embodiments;

FIG. 10 is a schematic illustration of image data acquired with the imaging system, according to various embodiments in a first position;

FIG. 11 is a detail view of the subject and the imaging system in a second position, according to various embodiments;

FIG. 12 is a schematic view of a position of the imaging system housing relative to a portion of the subject, according to various embodiments;

FIG. 13 is a schematic illustration of image data acquired with the imaging system, according to various embodiments in the second position;

FIG. 14A is a screenshot of a graphical representation of an instrument in a first position relative to a model of a portion of a subject, according to various embodiments;

FIG. 14B is a screenshot of a graphical representation of an instruments in a second position relative to the model of the portion of the subject, according to various embodiments;

FIG. 15A is a screenshot of a view of a model of a portion of a subject and a graphical representation of an instrument and a first position, according to various embodiments;

FIG. 15B is a screenshot of an image of the subject and a display of the graphical representation of the instrument in the first position, according to various embodiments;

FIG. 16A is a screenshot of a view of a model of a portion of a subject and a graphical representation of an instrument and a second position, according to various embodiments;

FIG. 16B is a screenshot of an image of the subject and a display of the graphical representation of the instrument in the second position, according to various embodiments;

FIG. 17A is a screenshot of a representation of a subject and a graphical representation of an orientation of an implant, according to various embodiments;

FIG. 17B is a screenshot including an image based upon image data of the subject and a graphical representation of an orientation of the implant, according to various embodiments;

FIG. 18A illustrates a model of a portion of the subject and a representation of at least two instruments and a vector therebetween, according to various embodiments; and

FIG. 18B illustrates a model of a portion of the subject and a representation of at least two instruments and a vector therebetween, according to various embodiments.

Corresponding reference numerals indicate corresponding parts throughout the several views of the drawings.

### DETAILED DESCRIPTION

Example embodiments will now be described more fully with reference to the accompanying drawings.

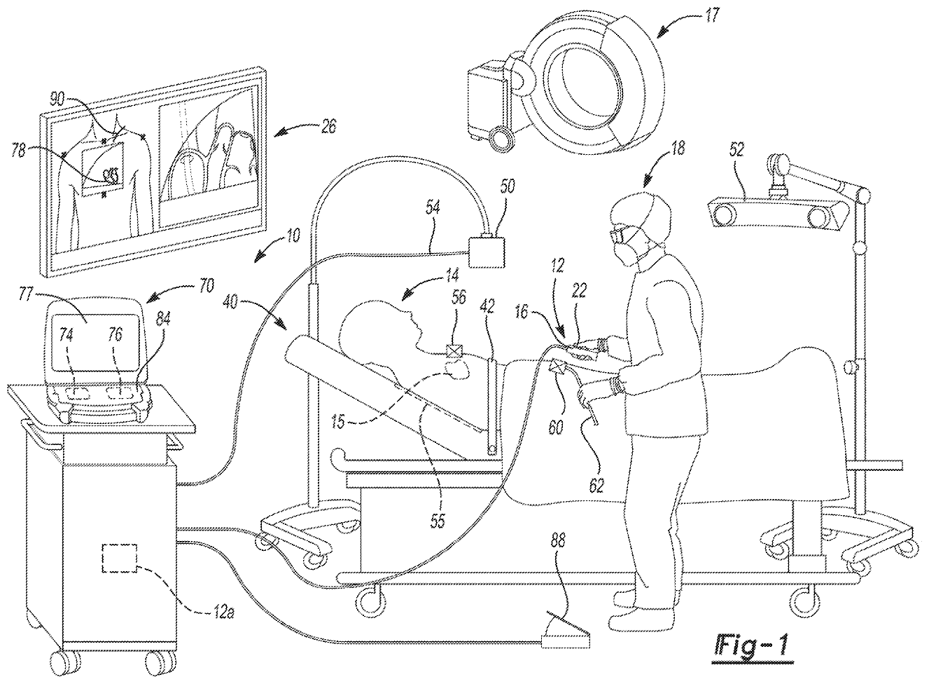

FIG. 1 is a diagram illustrating an overview of a navigation system 10 that can be used for various procedures. The navigation system 10 can be used to track the location of an item, such as an implant or an instrument, and at least one imaging system 12 relative to a subject, such as a subject 14 that may include a human patient and/or other living or non-living subject. It should be noted that the navigation system 10 may be used to navigate any type of instrument, implant, or delivery system, including: catheters, stylets, leads for cardiac rhythm management devices such as pacemakers, defibrillators, leadless pacemakers and delivery systems therefor, guide wires, arthroscopic systems, ablation instruments, stents, orthopedic implants, spinal implants, deep brain stimulation (DBS) probes, mechanical parts, Transesophageal Echocardiography (TEE), intra-cardiac echocardiography (ICE), etc. Non-human or non-surgical procedures may also use the navigation system 10 to track a non-surgical or non-human intervention of the instrument or imaging device. Moreover, the instruments may be used to navigate or map any region of the body. The navigation system 10 and the various tracked items may be used in any appropriate procedure, such as one that is generally minimally invasive or an open procedure.

The navigation system 10 can interface with or integrally include an imaging system 12 that is used to acquire pre-operative, intra-operative, or post-operative, or real-time image data of the patient 14. For example, the imaging system 12 can be an ultrasound imaging system (as discussed further herein) that has a tracking device 22 attached thereto. The tracking device 22 may be tracked with the tracking system to determine a pose of the imaging system 12. The pose may include an orientation (e.g., three or more degrees of freedom of orientation (e.g., yaw, pitch, and roll)) and/or a position (e.g., three degrees of freedom in physical space (e.g., x-axis, y-axis, and z-axis)). The tracking system 10 may determine appropriate pose information regarding the tracking device 22. The pose of the imaging system 12 can then be determined based on the tracked pose of the tracking device 22. In various embodiments, the tracking device 22 is fixed to or relative to the imaging system 12. The imaging system 12 may be used to generate image data to provide images for viewing with a selected display device 26, which may be any appropriate display device including an augmented and/or virtual reality display device worn/used by the user 18 such as those disclosed in U.S. Pat. App. Pub. No. US2018/0078316A1, incorporated herein by reference.

It will be understood that any appropriate subject can be imaged and any appropriate procedure may be performed relative to the subject. The navigation system 10 can be used to track various tracking devices, as discussed herein, to determine locations of the patient 14. The tracked poses of the patient 14 can be used to determine or select images for display to be used with the navigation system 10. The initial discussion, however, is directed to the navigation system 10 and the exemplary imaging system 12.

In the example shown, the imaging system 12 includes an ultrasound (US) imaging system with an US housing 16 that is held by a user 18 while collecting image data of the subject 14. It will be understood, however, that the US housing 16 can also be held by a stand or robotic system while collecting image data. The US housing 16 and included transducer can be any appropriate US imaging system 12, such as the M-TURBO® sold by SonoSite, Inc. having a place of business at Bothell, Washington. Associated with, such as attached directly to or molded into, the US housing 16 or the US transducer housed within the housing 16 is at least one imaging system tracking device 22. The tracking device 22 may be any appropriate tracking device such as an electromagnetic tracking device and/or an optical tracking device. The tracking devices can be used together (e.g., to provide redundant tracking information) or separately. Also, only one of the two tracking devices may be present. It will also be understood that various other tracking devices can be associated with the US housing 16, as discussed herein, including acoustic, ultrasound, radar, electrical impedance, and other tracking devices. Also, the tracking device 22 can include linkages or a robotic portion that can determine a location relative to a reference frame.

In various embodiments, a supplemental and/or secondary imaging system 17 may alternatively or also be present and/or used to generate image data of the patient 14. The secondary imaging system may include an O-arm® imaging device sold by Medtronic Navigation, Inc. having a place of business in Louisville, Colorado, USA, a C-arm imaging system, or other appropriate imaging system. The second imaging system can include those disclosed in U.S. Pat. Nos. 7,188,998; 7,108,421; 7,106,825; 7,001,045; and 6,940,941; all of which are incorporated herein by reference. The secondary imaging system, however, need not be present and the imaging system 12, which may be only the US transducer, may be the only imaging system to generate images to be displayed. Similarly, the US imaging system 12 need not be used and the secondary imaging system may be the only imaging system. As discussed herein, various other images may also be displayed such as an avatar 120 of the patient 14. The avatar 120 may be based on a model or average patient and/or other appropriate data or images. The secondary imaging system 17 may include a x-ray source and a x-ray detector that is operable to generate image date of the subject 14. The secondary imaging system 17 may also or alternatively be a computed tomography (CT), magnetic resonance imaging (MRI), etc.

The imaging system 12 may be tracked, as discussed above. Various tracking systems may include and/or require calibration of the imaging system. Thus, the pose of the tracking device 22 relative to a plane of the US imaging system may be determined and/or known. Various systems and methods are disclosed in U.S. Pat. Nos. 6,379,302; 6,669,635; 6,968,224; 7,085,400; 7,831,082; 8,320,653; 8,811,662; and 9,138,204 all of which are incorporated herein by reference. Briefly, the imaging system 12 may image a tracked jig having imageable portions at known poses relative to a portion of the jig engaging the imaging system 12. Thus, the tracked pose of the jig and the imaging system may be used to calibrate the imaging system to determine a pose of an imaged portion with the imaging system 12.

As illustrated in FIG. 1, the patient 14 can be positioned, including fixed, in a pose relative to a selected object, such as onto an operating table 40, but is not required to be fixed to the table 40. The table 40 can include a plurality of straps 42. The straps 42 can be secured around the patient 14 to fix the patient 14 relative to the table 40. Various apparatuses may be used to position the patient 40 in a static position on the operating table 40. Examples of such patient positioning devices are set forth in commonly assigned U.S. patent application Ser. No. 10/405,068, published as U.S. Pat. App. Pub. No. 2004/0199072 on Oct. 7, 2004, entitled “An Integrated Electromagnetic Navigation And Patient Positioning Device”, filed Apr. 1, 2003 which is hereby incorporated by reference. Other known apparatuses may include a Mayfield® clamp.

The navigation system 10 includes at least one tracking system. The tracking system can include at least one localizer. In one example, the tracking system can include an electromagnetic (EM) localizer 50. The tracking system can be used to track instruments relative to the patient 14 or within a navigation space. The navigation system 10 can use image data from the imaging system 12 and information from the tracking system to illustrate locations of the tracked instruments, as discussed herein. The tracking system can also include a plurality of types of tracking systems including an optical localizer 52 in addition to and/or in place of the EM localizer 50. When the EM localizer 50 is used, the EM localizer can communicate with or through a localizer communication 54 that may be wired or wireless. The EM localizer 50 may also include and/or take the form of an alternative pad or flat EM localizer 50.

The optical localizer 52 and the EM localizer 50 can be used together to track multiple instruments or used together to redundantly track the same instrument. Further, the coordinate systems of the two, or more, optical localizer 52 and the EM localizer 50, can be coordinated and registered to each other as is understood by one skilled in the art. Various tracking devices, including those discussed further herein, can be tracked and the information can be used by the navigation system 10 to allow for an output system to output, such as a display device to display, a position of an item. Briefly, tracking devices can include a patient or reference tracking device 56 to track the patient 14, an instrument tracking device 60 to track an instrument 62, and/or other appropriate tracking devices for one or more portions. Patient or reference tracking device 56 and instrument tracking device 60 may include those disclosed in U.S. Pat. Nos. 8,060,185 and 8,644,907, both incorporated herein by refence. The tracking devices allow selected portions of the operating theater to be tracked relative to one another with the appropriate tracking system, including the optical localizer 52 and/or the EM localizer 50. The reference tracking device 56 can also or alternatively be positioned on an instrument and positioned within the patient 14, such as within a heart 15 of the patient 14.

It will be understood that any of the tracking devices 22, 56, 60 can be optical or EM tracking devices, or both, depending upon the tracking localizer used to track the respective tracking devices. It will be further understood that any appropriate tracking system can be used with the navigation system 10. Alternative tracking systems can include radar tracking systems, acoustic tracking systems, ultrasound tracking systems, electrical impedance tracking systems, radio frequency beacon, and the like. Exemplary tracking systems include those disclosed in U.S. Pat. Nos. 7,676,268; 8,532,734; and 8,494,608, all incorporated herein by reference. Each of the different tracking systems can be respective different tracking devices and localizers operable with the respective tracking modalities. Also, the different tracking modalities can be used simultaneously as long as they do not interfere with each other (e.g., an opaque member blocks a camera view of the optical localizer 52).

An exemplary EM tracking system can include the STEALTHSTATION® AXIEM™ Navigation System, sold by Medtronic Navigation, Inc. having a place of business in Louisville, Colorado. Exemplary tracking systems are also disclosed in U.S. Pat. No. 7,751,865, issued Jul. 6, 2010 and entitled “METHOD AND APPARATUS FOR SURGICAL NAVIGATION”; U.S. Pat. No. 5,913,820, titled “Position Location System,” issued Jun. 22, 1999 and U.S. Pat. No. 5,592,939, titled “Method and System for Navigating a Catheter Probe,” issued Jan. 14, 1997, all herein incorporated by reference.

Further, for EM tracking systems it may be necessary to provide shielding or distortion compensation systems to shield or compensate for distortions in the EM field generated by the EM localizer 50. Exemplary shielding systems include those in U.S. Pat. No. 7,797,032, issued on Sep. 14, 2010 and U.S. Pat. No. 6,747,539, issued on Jun. 8, 2004; distortion compensation systems can include those disclosed in U.S. Pat. No. Ser. No. 10/649,214, filed on Jan. 9, 2004, published as U.S. Pat. App. Pub. No. 2004/0116803, all of which are incorporated herein by reference.

With an EM tracking system, the EM localizer 50 and the various tracking devices can communicate through an EM controller which may be incorporated in the workstation 70 and/or separate therefrom. The EM controller can include various amplifiers, filters, electrical isolation, and other systems. The EM controller can also control the coils of the EM localizer 50 to either emit or receive an EM field for tracking. A wireless communications channel, however, such as that disclosed in U.S. Pat. No. 6,474,341, entitled “Surgical Communication Power System,” issued Nov. 5, 2002, herein incorporated by reference, can be used as opposed to being coupled directly to the EM controller. The EM controller can be incorporated into a navigation processing system 70.

It will be understood that the tracking system may also be or include any appropriate tracking system, including a STEALTHSTATION® TRIA®, TREON®, and/or S7™ Navigation System having an optical localizer, similar to the optical localizer 52, sold by Medtronic Navigation, Inc. having a place of business in Louisville, Colorado. Further, alternative tracking systems may include an electro-potential and/or electrical impedance tracking system including those disclosed in U.S. Pat. No. 5,983,126, to Wittkampf et al. titled “Catheter Location System and Method,” issued Nov. 9, 1999, U.S. Pat. No. 8,494,613 to Markowitz et al. issued Jul. 23, 2013; U.S. Pat. No. 8,494,608 to Markowitz et al. issued Jul. 23, 2013; and/or U.S. Pat. No. 8,532,734 to Markowitz et al. issued Sep. 10, 2013, all of which are hereby incorporated by reference. Other tracking systems include an acoustic, radiation, radar, etc. tracking or navigation systems.

The navigation system 10 can include a navigation processing unit or module 74 that can communicate or include a navigation memory 76, which may be included in the navigation processing system 70. The navigation processing system 70 may further include a display device 77. The navigation processing unit 74 can include a processor (e.g., microprocessor, a central processing unit, etc.). In various embodiments, the navigation processing unit may execute instructions to determine one or more poses of the tracking devices based on signals from the tracking devices. The navigation processing unit 74 can receive information, including image data, from the imaging system 12 and tracking information from the tracking systems, including the respective tracking devices and/or the localizers 50, 54. Image data can be displayed as an image 78 on the display device 26. The display device may be separate from and/or integrated into the navigation system, thus, the display device 26 may include or be the display device 77. The navigation system 70 can include appropriate input devices, such as a keyboard 84. It will be understood that other appropriate input devices can be included, such as a mouse, a foot pedal 88 or the like which can be used separately or in combination. Also, all of the disclosed processing units or systems can be a single processor module (e.g., a single central processing chip) that can execute different instructions to perform different tasks.

An image processing unit or module can process image data from the imaging system 12 and a separate first image processor 12a may be provided separate, such as with the navigation system 70, can be provided to process or pre-process image data from the imaging system 12. The image data from the image processor can then be transmitted to the navigation processor 74. It will be understood, however, that the imaging systems need not perform any image processing and the image data can be transmitted directly to the navigation processing unit 74. Accordingly, the navigation system 10 may include or operate with a single or multiple processing centers or units that can access single or multiple memory systems based upon system design.

In various embodiments, the imaging system 12 can generate image data that may be used to compose the image 78. For example, x-ray projections and/or MRI or CT image data slices may be used to generate an image for display with the selected display device 26, 77. The image and/or image data may define an image space that can be registered to a patient space or navigation space that is defined by and/or relative to the patient 14. In various embodiments, the position of the patient 14 relative to the imaging system 12 can be determined by the navigation system 10 with the patient tracking device 56 and the imaging system tracking device(s) 22 to assist in and/or maintain registration. Accordingly, the position of the patient 14 relative to the imaging system 12 can be determined.

Manual or automatic registration of the image space to the subject space can occur. In various embodiments, the registration can occur by matching fiducial points in image data with fiducial points on the patient 14. Registration of image space to patient space allows for the generation of a translation map between the patient space and the image space. According to various embodiments, registration can occur by determining points that are substantially identical in the image space and the patient space. The identical points can include anatomical fiducial points or implanted fiducial points. Exemplary registration techniques are disclosed in U.S. patent application Ser. No. 12/400,273, filed on Mar. 9, 2009, now published as U.S. Pat. App. Pub. No. 2010/0228117 and in U.S. Pat. No. 9,737,235, issued Aug. 22, 2017, both incorporated herein by reference.

According to various embodiments, the imaging system 12 can be used with an un-navigated or navigated procedure. In a navigated procedure, a localizer and/or digitizer, including either or both of an optical localizer 52 and/or an electromagnetic localizer 50, 55 can be used to generate a field and/or receive and/or send a signal within a navigation domain relative to the patient 14. The navigated space or navigational domain relative to the patient 14 can be registered to the image 78. Correlation, as understood in the art, is to allow registration of a navigation space defined within the navigational domain and an image space defined by the image 78. The patient tracker or dynamic reference frame 56 can be connected to the patient 14 to allow for a dynamic registration and maintenance of registration of the patient 14 to the image 78.

Once registered, the navigation system 10 with or including the imaging system 12, can be used to perform selected procedures. Selected procedures can use the image data generated or acquired with the imaging system 12. Further, the imaging system 12 can be used to acquire image data at different times relative to a procedure. As discussed herein, image data can be acquired of the patient 14 prior to the procedure for collection of automatically registered image data or cine loop image data. Also, the imaging system 12 can be used to acquire images for confirmation of a portion of the procedure. Thus, image data may be acquired at any appropriate time and may be registered to the patient 14.

Registration and navigated procedures are discussed in U.S. Pat. No. 8,238,631, incorporated herein by reference. Upon registration and tracking of the instrument 62, a graphic representation 90 (e.g., an icon, indicium, animation or other or visual representation) may be displayed relative to, including overlaid (e.g., superimposed) on, the image 78. The image 78 may be any appropriate image and may include one or more 2D images, such as 2D images that are acquired at different planes. Images may also be a 3D image, or any appropriate image as discussed herein.

In addition to registering the subject space to the image space, however, the imaging plane of the US imaging system 12 can also be determined. By registering the image plane of the US imaging system 12, imaged portions can be located within the patient 14. For example, when the image plane is calibrated to the tracking device(s) 22 associated with the US housing 16 then a position of an imaged portion of the heart 15, or other imaged portion, can also be tracked as disclosed in U.S. Pat. Nos. 6,379,302; 6,669,635; 6,968,224; 7,085,400; 7,831,082; 8,320,653; 8,811,662; and 9,138,204 all of which are incorporated herein by reference.

Once the patient 14 is in condition for a procedure, the patient 14 may be positioned on the table 40, as noted above. The display 26 and/or the display 77 may display various information regarding the patient 14 and/or other information selected by the user 18. As noted above, the display 26 may illustrate the image 78 that may be acquired with the imaging system 12, 17. As discussed herein, the imaging system according to any appropriate embodiment may be used to acquire image data that may be used to generate the image 78. Thus, discussion of any single imaging system is not intended to limit the scope of the subject disclosure unless specifically disclosed to be so limited. The image 78 may also be displayed on the display 77. Also as noted above, the instrument 62 may be tracked and the graphic representation 90 of the instrument 62 may be displayed on the display 26, such as relative to the image 78. In addition, and/or alternatively, the graphic representation 90 may also be displayed relative to a patient avatar 120.

The imaging system 12, 17 may be used to generate image data of the subject 14 to allow for generation of the image 78. The image 78 may be based upon image data that is acquired at any appropriate time, such as prior to a procedure, during a procedure, or at any selected time. As is understood in the art, the imaging system 12, 17 may generate an image that is registered to the subject 14, as discussed above. Thus, an image may be generated to allow for illustration of a pose of the instrument 62 relative to the image 78 that may not be a real time image of the subject 14. Therefore, the pose of the instrument 62 may be displayed relative to the image 78 based upon tracking of the instrument 62 and of the subject 14.

As discussed further herein, the patient avatar 120 may also be displayed relative to the graphical representation 90 of the instrument 62. The avatar 120 of the subject 14 may be displayed based upon a predetermined or measured dimension of the subject 14. The graphical representation 90 of the instrument may be superimposed (e.g., overlaid) on the avatar 120.

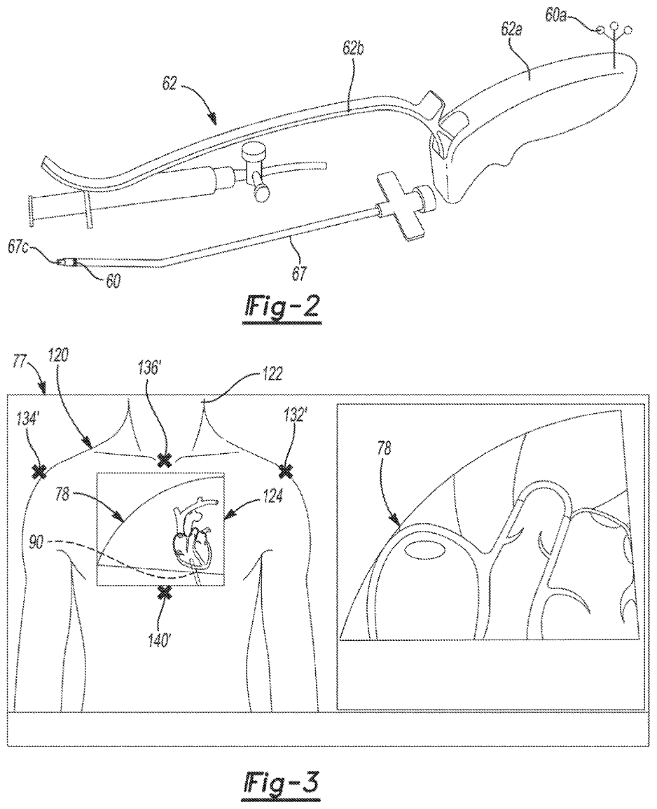

The instrument 62 may include various instruments such as a tunneling tool, such as a substernal tunneling tool, as illustrated in FIG. 2. The tunneling tool 62 may include a grip or handle portion 62a and a guide portion 62b. A tunneling portion 67 may be moved with the tool 62 and relative to the guide portion 62b. The tunneling portion 67 may be tracked, such as at a distal tip 67c thereof with the instrument tracking device 60. The tunneling portion 67 may be moved with the handle portion 62a and relative to the guide 62b. The instrument tracking device 60 may be positioned at the distal tip 67c to allow for direct tracking of the distal tip 67c of the tunneling portion 67. In various embodiments, however, an instrument tracking device 60a may be positioned at a point away from the distal tip 67c. Dimensions between the instrument tracking device 60a and the distal tip 67c may be used to determine the pose of the distal tip 67c. The tunnelling portion 67 may include a sheath extending over at least a portion of a tunneling rod. The sheath may be slidably disposed over the tunneling rod such that when the tunneling portion is positioned, the tunneling rod may be retracted thereby leaving the sheath in place within the substernal space. Further, a tracking device, according to various embodiments, may be positioned on the guide portion 62 to determine a pose of a portion, such as the distal tip 67c, of the tunneling portion 67 if/when the pose of the guide portion 62b is known relative the tunneling portion 67.

The tunneling tool 62 is configured to be positioned between the heart 15 of the subject and the sternum 14s of the subject 14. It is understood, however, that the tool 62 and/or portions thereof may be used to be positioned laterally to the heart, within the pleura, within the pericardium, epicardial, posterior of the heart, inferior of the heart. The tunneling portion 67 and the tip 67c is configured to be positioned transcutaneous into the subject 14 to be positioned between the heart 14 and the sternum 14s. Thus, the incision(s) to allow access to a sub-sternal region in the subject 14 may generally be about 5% to about 300%, including about 5% to about 100%, and further including about 5% to about 50%, larger than the tunneling portion 62b. The tunneling portion 62b may, therefore, be positioned through the incision and tunnel between the sternum 14s and the heart 15. As discussed herein, the tunnel, which may include placement of the sheath 67, may allow for positioning of various implants such as a lead 154.

The instrument tracking device 60 may be an appropriate tracking device such as including a plurality of coils over the tunneling tool 62. In various embodiments, the coils may include those disclosed in U.S. Pat. No. 8,644,907, incorporated herein by reference. In various embodiments, the coils may include one or more coils that are wrapped around the tunneling tool 62. The coil may be wrapped at selected angles relative to one another to allow for a pose determination at a plurality of degrees of freedom. The coils may sense and/or transmit a field wirelessly and/or through a wired connection. Thus, the tunneling tool 62 may be tracked within the tracking system as discussed above.

The tracking of the tunneling tool 62 allows for a determination of the pose of at least the distal tip 62c and/or other portions of the tunneling tool 62. The tracked pose may be displayed relative to the image 78 and/or the avatar 120, as discussed above and further herein. For example, as illustrated in FIG. 3, a tracked pose may be illustrated. The user 18 may then view and/or determine the pose such as relative a mid-line position. For example, the sternal tunneling tool 62 may be used to position the sheath 67 relative to the heart 15 of the subject 14. The general position of the instrument 62 may be displayed with the graphical representation 90 relative to the avatar 120 and/or the image 78.

Although the systems and methods described herein refer primarily to embodiments in which the instrument 62 is a tunneling tool, it should be understood that the instrument 62 may additionally or alternatively include other one or more kinds of instruments, such as a lead (e.g., cardiac lead), or a sheath (e.g., arranged over a tunneling tool or lead), a sheath and a tunneling tool in combination, or a tunneling tool and a lead (e.g., cardiac lead) in combination. In some embodiments, the instrument 62 includes one or more instruments that helps enable insertion of a lead (e.g., cardiac lead) to a desired target location. The instrument 62 can generally include any suitable distal portion, and the distal portion of the instrument 62 can be controlled (e.g., moved) with a handle.

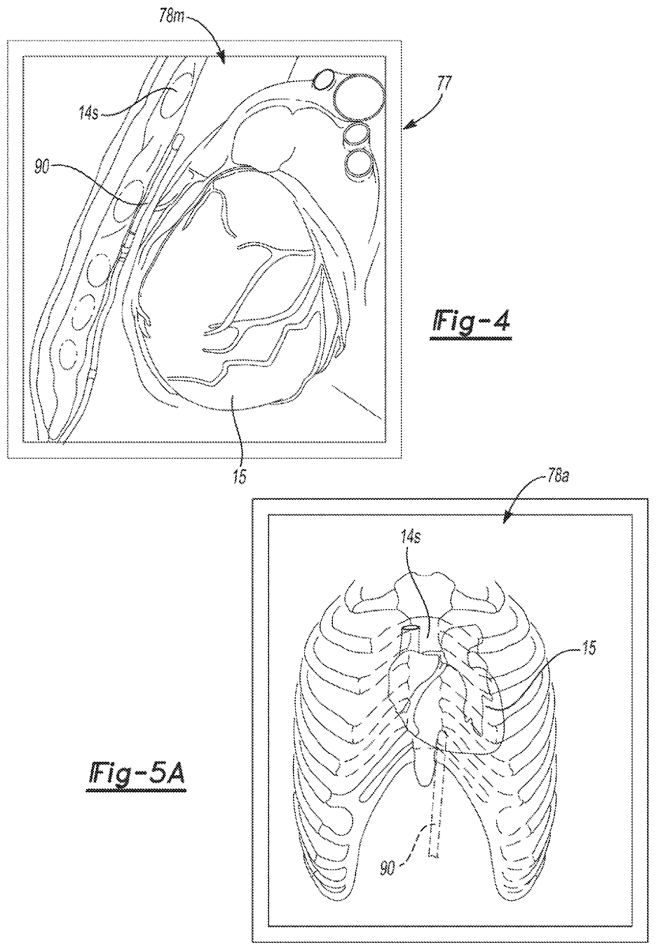

With continuing reference to FIG. 3 and additional reference to FIG. 4, the image 78 may be displayed on the display device 77. The image 78 may include a medial to lateral view 78m. The medial to lateral view 78m may be used to illustrate the graphical representation 90 of the instrument 62. The graphical representation 90 may include a pose relative to the heart 15 of the subject 14 of the instrument and/or any appropriate portion thereof (e.g., tunneling portion 67, lead, etc.) and/or the lead to be implanted or as implanted. Thus, the user, such as the surgeon 18 may understand the pose relative to the heart 15. For example, a distance away from the heart and/or adjacent to a sternum 14s may be determined. For example, it may be selected to position the instrument 62 as near the heart 15 as possible without intersecting tissue of the heart 15.

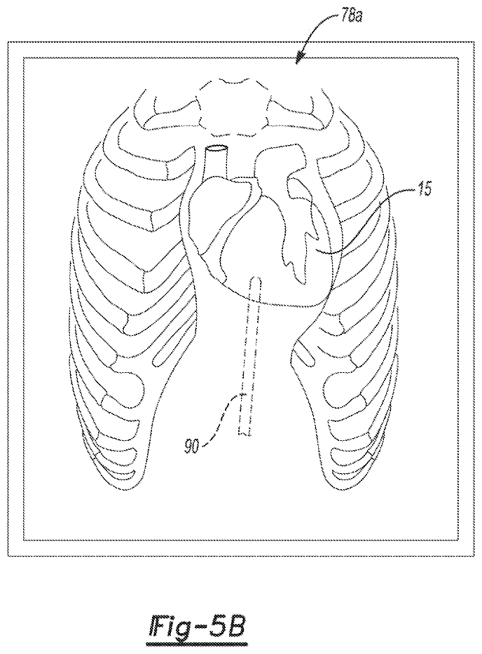

Similarly, the image 78 may include an anterior to posterior image 78a, as illustrated in FIG. 5. Again, the graphical representation 90 of the instrument may be displayed to illustrate a medial to lateral view of the instrument 62 relative to the subject 14 and various anatomical features, such as the heart 15. The graphical representation 90 may also be displayed relative to other tissues and/or structures, such as the sternum 14s. Again, the user 18 may then understand that the medial and/or lateral position of the instrument 16 by viewing the graphical representation thereof.

The patient avatar 120 may be illustrated on a selected display, as illustrated in FIGS. 1-3, such as the display 26. The patient avatar 120 may be based on a general avatar that includes various features of the patient 14, as noted herein, but is sized to the current and specific patient. The avatar 120 may be illustrated as a two-dimensional (2D) image and/or a three-dimensional (3D) image. The avatar 120 may be any appropriate shape to assist in identifying various positions and relative positions of portions of the patient 14. The avatar 120, according to various embodiments, is sized to the specific patient as disclosed herein.

The patient avatar 120 may generally illustrate a portion of the patient 14 and may be sized relative to the patient 14. For example, the patient avatar may illustrate an inferior portion of a neck 122, a pectoral region 124, and other appropriate regions of the patient 14. The patient avatar 120 may be displayed on the display device 26 to illustrate a general representation of the patient 14 without requiring an image acquisition of the same portion of the patient 14. Therefore, the patient avatar 120 may provide a general roadmap or indication of the position of various tracked portions, as discussed further herein. The avatar 120 may however, as discussed herein, be morphed to match a relative size of the patient 14 to provide an indication of relative anatomical portions of the patient 14 and a pose of the tracked portions, such as the instrument 62, relative thereto.

As discussed above, the tracked pose of the instrument 62 may be illustrated relative to the image 78 and/or the avatar 120. The user 18, therefore, may understand the pose of the instrument 62. The pose of the instrument 62 may be illustrated relative to the image 78 and/or the avatar 120 without requiring a constant acquisition of image data of the subject 14 including the instrument 62. Thus, a real time pose of the instrument 62 may be known to the user 18 and displayed on the display device, without using real-time image data. This may reduce exposure of the subject 14, such as to x-rays, and provide other advantages such as reducing operating time by not requiring movement of an imaging system. Further, the user 18 or other individual need not operate the imaging system to acquire real time image data of the subject 14 to determine the pose of the instrument 62 within the patient 14. The tracked pose of the instrument 62 with the tracking device 60 may be used to determine and illustrate the pose of the instrument 62. As noted above, the image data may be registered to the subject 14 and the registration may be maintained such as with of the patient tracker 56.

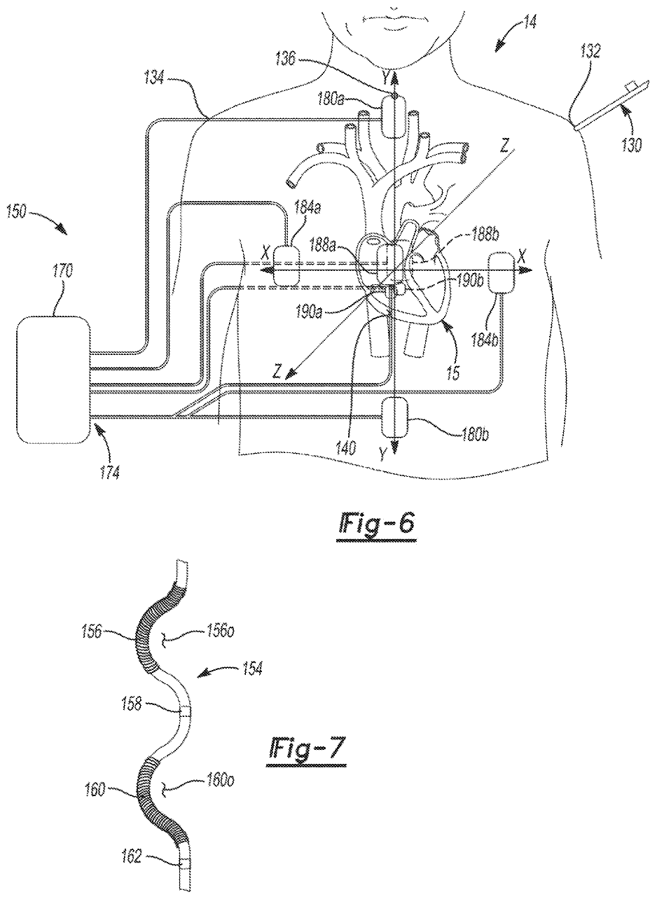

With continuing reference to FIGS. 1-5 and additional reference to FIG. 6, the avatar 120 may be generally sized to the patient 14 according to one or more appropriate procedures. The avatar 120 may be sized or morphed in real time to match relative dimensions of the patient 14. The relative positions of the anatomic portions may include a relative position of a suprasternal notch and an exterior shoulder or inferior jugular vein, or any other suitable anatomical location(s).

In various embodiments, for example, the user 18 may provide or include information regarding the patient 14. As noted above, the navigation processing system 70 may include various inputs that allow the user 18 to input information. Therefore, the user 18 may input information such as a height, a weight, and/or a sex of the patient 14. The input information is used by the processor module 74 to execute selected instructions to size the patient avatar 120 to the actual patient 14. The resizing of the avatar 120 may be based upon recalling of a selected or known predetermined size from a database, reconfiguring or resizing the patient avatar 120 based upon the input information, or other appropriate information. Therefore, the avatar 120 may be sized to substantially match the patient 14 without acquiring an image of the patient 14 for display on the display device 26.

In addition or alternatively thereto, the user 18 may also identify or size the avatar 120 to the patient 14 with a tracked indicator or tracked member 130. For example, the tracked indicator 130, which may be an indicator or probe usable to touch a surface of the patient 14, may be moved by the user 18 to various predetermined positions on the patient 14 as indicated on the avatar 120. The tracked indicator 130 may be tracked with any appropriate tracking system, including those discussed herein according to various embodiments such as the localizer system of U.S. Pat No. 5,983,126, hereby incorporated by reference. The user 18 may move the tracked indicator 130 to various points, such as the exterior shoulder portions 132′, 134′ as illustrated by the icons 132 and 134. Additionally, the user 18 may move the tracked indicator 130 to other portions such as a suprasternal notch 136 and a sternum 140 of the patient 14 or outer boundary positions. It is understood that the tracked indicator 130 may be moved to any appropriate position and the above-noted positions are merely exemplary. Nevertheless, based upon the tracked position of the tracked indicator 130, the avatar 120 may be sized to morph the identified points to a representation of the patient 14 for display on the display device 26. Also, the points on the avatar 120 may be registered to the patient 14 by identifying them in the patient space relative to the patient 14 and identifying them on the avatar 120.

The avatar 120 may also be morphed to actual measurements taken of the patient 14. For example, a distance between the shoulder points 132′, 134′ on that the patient 14 may be measured, such as with a tape measure. The measured distance may be input within the navigation processing system at 77. The avatar 120 may be sized based upon the measurements.

Regardless of the technique, the avatar 120 may be matched or morphed to substantially mimic the shape of the patient 14. The avatar 120, as discussed further herein, may then be used as an illustration of a pose of the tracked instrument, such as the instrument 62, during a procedure on the patient 14. Thus, the avatar 120 may provide a representation of a position of the tracked instrument 62 without requiring an image acquisition of the patient 14, such as with a fluoroscopic imaging system. The avatar 120, as it is morphed to the specific patient may or will be displayed as a patient specific avatar. Thus, the avatar 120 may include general information, but is displayed and registered to the patient 14 as a patient specific avatar 120 including relevant locations and relative positions of various portions of the patient 14. The specific patient avatar illustrates the relative positions of the anatomy of the patient 14 that is the current patient. For example, the position of the heart 15 relative to the suprasternal notch.

The avatar 120 may be registered to the patient 14 in any appropriate manner, including those discussed above. In various embodiments, for example, when the tracked indicator 130 is tracked to determine positions of the patient 14 for marking or changing the avatar 120. The patient 14 after the various points are determined in both the patient 14 and the avatar 120 may be registered thereto. Additionally, and/or alternatively, the patient tracker 56 may be positioned on the patient 14 at an appropriate position. The position of the patient tracker 56 may be at the suprasternal notch 136′ (FIG. 3) which relates to a point on the avatar 120, such as the suprasternal notch 136. The position of the suprasternal notch 136 in the avatar 120 may be determined or known to be the position of the patient tracker 56 to allow for registration of the avatar 120 relative to the patient 14. Also, the patient tracker 56 may maintain registration even when the patient 14 moves relative to the localizer(s). It is understood that any appropriate number of the patient trackers 56 may be utilized, including each may be used for registration and/or more than one of the patient trackers 56 may be used.

The localizers, including the EM localizer 58, may also or alternatively be fixed or positioned relative to the patient 14 in an appropriate manner. For example, the localizer 58 may be fixed relative to the table 14 such that it is immovable during the procedure. Thus, the field generated with the EM localizer 58 can be at a known position relative to the patient 14 and allow for a registration of the avatar 120 to the patient 14 based upon the known position of the EM localizer 58 relative to the patient 14.

With continuing reference to FIG. 6, as noted above, an electro-potential (EP) or electrical impedance tracking system 150 may also be provided. The EP tracking system 150 may allow for tracking an instrument, such as the instrument 62 as noted above. Further, the EP tracking system 150 may track additional instruments which may include an implant, such as a lead 154, as illustrated in FIG. 7. The lead 154 may include various portions, such as at least a first coil electrode 156, and a ring electrode 158. The lead 154 may also include additional electrodes such as a second coil electrode 160 and a second ring electrode 162. The lead 154 may be elongated and positioned within the subject 14 with of the sheath 67. The sheath 67 may then be withdrawn to leave the lead 154 in place. As discussed further herein, the tunneling instrument 62 may be used to position that the sheath 67 relative to the heart 15. The lead 154 may then be passed through the sheath 67 within the patient 14. The sheath 67 may then be removed.

The electrodes of the lead 154 may be used to determine at least an orientation of the coil electrodes 156, 160 relative to the subject 14. The EP tracking system 150 may include a plurality of electrode patches that are positioned on the subject 14. The patches may be electrodes that inject a current into the subject 14 and the electro-potential may be sensed at the electrodes of the lead 154 and a signal based on the sensed electrical potential may be passed to the navigation system to assist in determining an orientation and possibly a pose of the lead 154.

The EP tracking system 150 can include a control or driving unit 170 that includes one or more input or output connectors 174 to interconnect with a plurality of current conducting or drive patches connected directly with the patient 14. Current patches can include patches to create three substantially orthogonal voltage or current axes within the patient 14. For example, a first y-axis patch 180a and a second y-axis patch 180b can be interconnected with the patient 14 to form a y-axis (such as an axis that is generally superior-inferior of a patient as illustrated in FIG. 7) with a conductive path such that the conducted current establishes a voltage potential gradient substantially along this axis and between the patches 180a and 180b. A related y-axis current may flow from the first y-axis patch 180a to the second y-axis patch 180 substantially along the y-axis. Likewise, a first x-axis patch 184a and a second x-axis patch 184b can be connected with the patient 14 to create a x-axis (such as an axis that is generally medial-lateral of a patient) with a voltage gradient substantially along the x-axis between the patches 184a and 184b and a corresponding x-axis current flowing between patches 184a and 184b. Finally, a first z-axis patch 188a and a second z-axis patch 188b can be connected with a patient 14 to create a z-axis (such as an axis that is generally anterior-posterior of a patient) with a voltage potential gradient substantially along the z-axis between the patches 188a and 188b with a corresponding z-axis current flowing between the patches 188a and 188b. The three axes are generally formed to have an organ or area of interest at the common intersection or origin of each of the axes x, y, z. Accordingly, the patches 180, 184, 188 can be positioned on the patient 14 to achieve the selected placement of the axes x, y, z relative to the patient 14, such as at or near the heart 15. Each of the patches 180, 184, 188 can be interconnected with the PSU input/output (I/O) box 170, via a wire connection or other appropriate connection at the ports 174.

In addition, reference patches can be interconnected with the patient 14 for reference relative to the patient 14. The reference patches can include a first reference patch 190a and a second reference patch 190b. The placement of the reference patches 190a, 190b can be any appropriate position on the patient 14, including those discussed further herein according to various embodiments.

The current applied between the related patches generates a small- or micro-current, which can be about 1 microampere (μA) to about 100 milliamperes (mA), in the patient along the axis between the respective patch pairs. The induced current can be of a different frequency for each of the related patch pairs to allow for distinguishing which axis is being measured. The current induced in the patient 14 will generate a voltage gradient across different portions, such as the heart, that can be measured with a position element. The position element can be an electrode, such as the coil electrode 156, 160. The sensed voltage can be used to identify a position along an axis (whereby each axis can be identified by the particular frequency of the current being measured) to generally determine a position of an electrode along each of the three axes. Although a voltage can be sensed, an impedance can also be calculated or measured to determine a location in a similar manner. It will be understood, that a sensing of voltage will not eliminate other possible measurements for position determination, unless specifically indicated.

The PSU I/O box 170 can be interconnected with the workstation 70, via a connection or data transfer system. The data transfer system can include a wire transmission, wireless transmission, or any appropriate transmission. The workstation 70 can receive signals, which can be analog or digital signals, regarding voltages sensed by the reference patches 190a, 190b and electrodes on the instrument 62, 154. The signals can be used to determine a relative location of the instrument 62, 154 and to display the determined relative location on the display device 26. The display device 26 can be integral with or separate from the workstation 70. In addition, various interconnected or cooperating processors and/or memory can be provided to process information, each may be a part of the workstation 70 or separate therefrom. The processors can process the signals from the patches 180-190 and instrument 62, 154 to determine the position of the instrument 62, 154, display the determined positions or other data on the display device 26.

The multiple driving or voltage patches 180-188 are used to conduct current in the patient to create voltage potentials within the patient 14 that can be sensed by electrodes that are positioned on or within the patient 14. It will be understood that the driving patches 180-188 can be positioned on the patient 14 at any appropriate locations, such as the locations described with the Local Lisa™ position sensing unit previously provided by Medtronic, Inc. of Minneapolis, Minn., USA. The PSU I/O box 170, can create voltages and generate a small current along the axes between the related patches. The current generated can include different frequencies along the different x, y, and z axes to distinguish the x, y, and z-axes.

In various embodiments, a calculated impedance or sensed voltage at one or more electrodes, such as of the instrument and/or the lead 154 can be used to determine a location of the electrode of the instrument 62, 154 relative to a selected reference, such as reference patch 190a, 190b. The reference patches 190a, 190b can be positioned at any appropriate position on the patient 14. For example, the reference patches can be used to reorient or register position and/or image data to the patient 14. Therefore, the reference patch 190a, 190b can be a substantially fixed reference patch for reference regarding the voltage generated by the EP tracking system 150. Accordingly, it will be understood that the position of an electrode, such as of an instrument, can be determined based upon a relationship of Ohms Law by determining an impedance or measuring voltage within the patient 14.

Reference patches can also be used to measure a voltage drop of the tissue patch interface. Patches driven with current have a voltage drop across the electrode tissue interface. Using raw unreferenced voltage introduces measurement error which is eliminated by use of a reference. The reference electrodes can be used to measure the voltage drop.

The imaging system 12, as discussed above, may be the US imaging system. The US imaging system 12 may image of the subject 14 by movement and/or placement of the US imaging system 12 as illustrated in FIG. 7. Further, the US imaging system 12 may include the housing 16 that is tracked with of the tracker 22. The tracker 22 may be any appropriate tracker, such as an EM tracker, an optical tracker, or the like. The optical tracker may be tracked by the optical localizer 52 and the EM tracker may be tracked by EM localizer 50. The tracker may allow for a determination of a pose of the housing 16. As discussed above, the US imaging system 12 may generate image data or acquire image data in a plane relative to the housing 16. An ultrasound imaging plane 200 may be used to acquire image data of the subject 14 when positioned relative to the subject 14.

The US housing 16 may be moved relative to the subject 14 to image the subject or a portion of the subject, such as acquiring an image at a parasternal view of the heart 15. The parasternal view of the heart 15 acquired with of the US imaging system 12 may be one that is generally from an anterior side of the patient 14. As illustrated in FIG. 9, the US housing 16 may be positioned a distance 204 from a surface of the heart 15s. The distance 204 may be known based upon a predetermined knowledge of the plane 200. Therefore, a distance from the US housing 16 may be known by the navigation system 10. Further, as discussed above, positions of portions within the plane 200 may be determined based upon a calibration of the plane 200 relative to the tracking device 22. With reference to FIG. 10, therefore, the ultrasound image data 210 may be analyzed to identify various features, such as a septum 214′ and an aortic valve 216′ in the ultrasound data 210. These features may also, therefore, be determined in the patient space due to the tracked position of the US housing 16 to be able to identify the aortic valve 216 and the septum 214.

At least because the US housing 16 is tracked with the tracking device 22, the known distance 204 of the US housing 16 relative to the surface 15s of the heart 15 may be known. Further positions of other portions may also be known, such as the septum 214 and that the aortic valve 216. This allows the image 78 to be displayed relative to the subject 14 at a known space. Further dimensions of various features of the anatomy of the subject 14, such as that of the heart 15, may be known and may be modeled or used to generate a model of the heart 15.

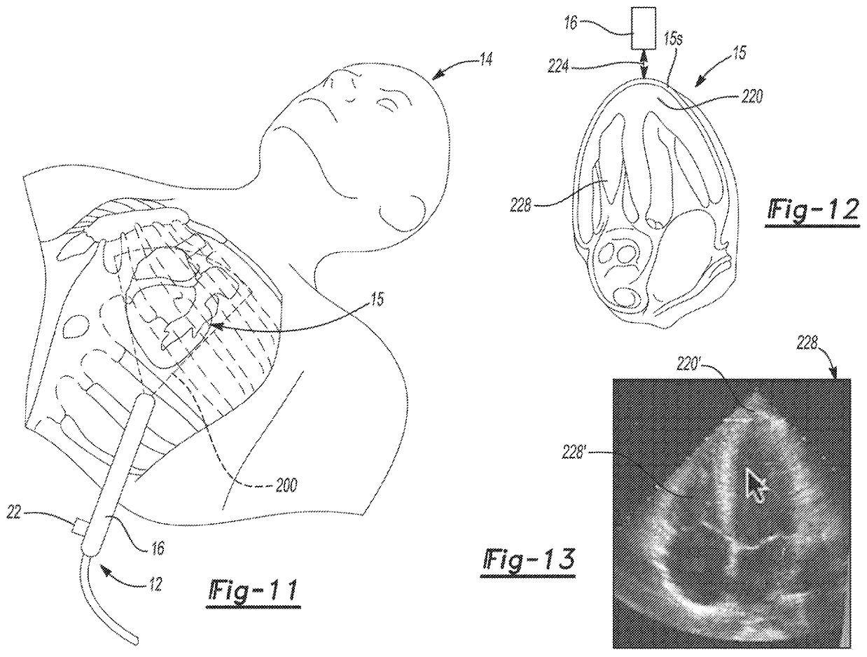

Additional image data of the subject 14 may be acquired with the ultrasound housing 16 as it may be moved relative to the subject 14. For example, as illustrated in FIGS. 11, 12, and 13 the ultrasound housing 16 to may be moved relative to the heart 15 such that it is generally near an apex of the heart 220. Again, the distance of the ultrasound housing 16 may be a distance of 224 from the surface 15s of the heart. The distance may be known due to the predetermined imaging modality of the US imaging system 12. Again, ultrasound image data may be acquired including ultrasound image data 228 as illustrated in FIG. 13. The image data may include various features such as the apex of the heart 220′ and a right ventricle 228′ respectively to the same features of the heart 15 including the apex of the heart 220 and a right ventricle 228′. Again, the features may be known in the image data relative to the tracked pose of the US housing 16 due to the tracking of the US housing 16.

As the US housing 16 is tracked to acquire image data of the various features of the heart 15, dimensions of various portions (e.g., lungs, diaphragm, esophagus) relative to each of the portions of the heart may be determined in the image data based on predetermined position relative to the US housing 16. The image data may be combined to generate a model of the heart 15. The image data may be used to identify and/or morph a surface of an atlas heart based upon the imaged heart 15 of the subject 14. Therefore, the US housing 16 may be moved relative to the subject 14 and the known dimensions of the spacing of the probe 16 relative to the surface in the image data may be used to morph and/or define a surface of an atlas heart to the heart 15 of the subject 14. In a similar manner, a pre-acquired image may be registered to the image data acquired with the US imaging system 12 due to the tracked pose of the US housing 16 and the position of the image data acquired with of the US housing 16.

As discussed above and further herein, therefore, the various image data, a model, or registered pre-acquired image may be displayed with the display device 26. The tracked pose of the instrument 62, 154 may also be displayed relative thereto. The image displayed on the display device 26, therefore, may be generated completely without any image data from an x-ray or fluoroscopic imaging system. The image data may be acquired with the nonionizing imaging system, such as the US imaging system 12. Nevertheless, the tracked pose of the US housing 16 allows for the image data be acquired of the subject at a known pose relative to the subject. Thus, the image data acquired with the US imaging system 12 may be used to illustrate a tracked pose of the instrument 62, 154 relative to an image, model, or registered pre-acquired image data to allow the user 18 to view and/or understand the pose of the tracked instrument 62, 154 relative to the subject 14.

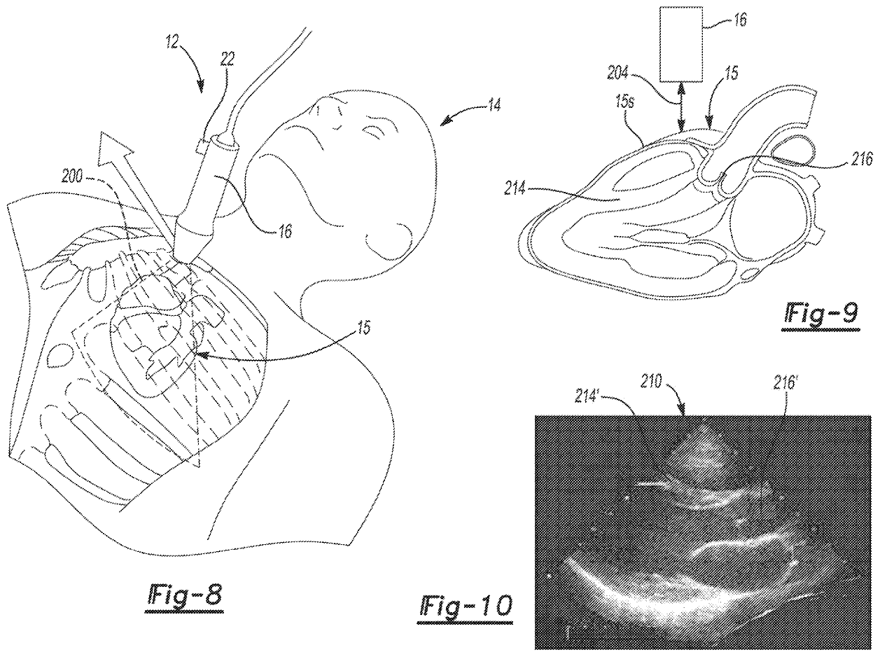

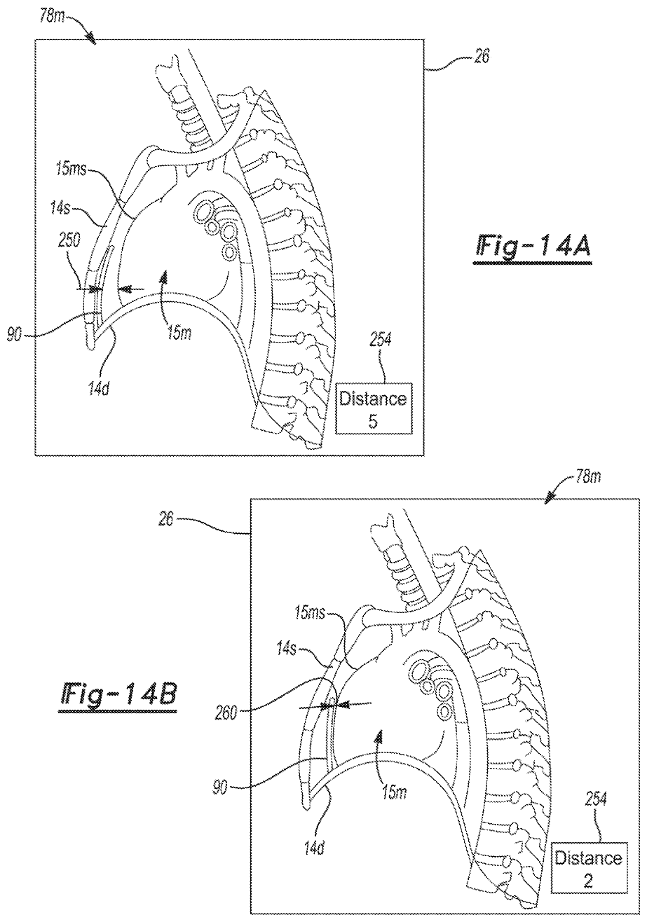

With continuing reference to FIGS. 1-13, and additional reference to FIGS. 14A and 14B, the instrument 62, 154 may be tracked relative to the patient 14 which may be displayed such as within the graphic representation 90 relative to selected portions including the avatar 120, the image 78, and/or an atlas or model 78m. The atlas 78m may be based upon the selected information, such as based on a plurality of images or image data of patients that have been averaged and portions thereof identified. For example, the atlas 78m may be a model based on the image data from a plurality of images. The atlas 78m (also referred to as a model) may have selected portions therein that are identified. In various embodiments, the atlas 78m may include the following identified locations, as is understood by one skilled in the art.

With reference to FIGS. 10 and 13, the images 210, 228 is an example of one image or a set of images that can be used to build the atlas 78m. The atlas 78m may also be generated using the patient's pre-procedure imaging like a CT or MRI. The image 78 may be obtained with a selected imaging system, including the US imaging system 12, as discussed above. A patient specific anatomy model may be generated using multiple 2D/3D views/planes stitched together, for example to reconstruct the endocardium of the heart. To facilitate building the atlas 78m, a clinician may identify various relevant portions or locations within the image 210, 228 Alternatively, the atlas 78m may be built only from images of the anatomy of the patient 14, and the portion(s) or location(s) of interest applied to the atlas based on identification within the images of the patient's own anatomy, and/or based on a compilation of images from other patients' anatomy and an estimated and/or average position of the portion(s) or location(s) of interest within the compilation of images. The atlas 78m may also be morphed to actual measurements taken of the patient 14.

In addition to providing more than one view of the heart 15 of the patient 14, the two images 210, 228 may also assist in registration to the model 78m. Thus, the identification of anatomy in the one or both of the images 210, 228, may be assisted by the registration of more than one image relative to the model or atlas 78m. As discussed above, the ultrasound imaging system 12 is registered to the patient 14, thus the images acquired with of the imaging system 12 may also be registered to the patient 14. The registration of the images to the patient may be based upon the tracking of the imaging system 12, such as with the imaging system tracking device 22 and the tracking of the patient 14, such as with the patient tracking device 56. Once the image 210, 228 and/or model 78m is registered to the patient 14, the instrument 62, 154 may be tracked relative to the patient 14 and its pose may be displayed relative to the image 210, 228 and/or model 78m. As discussed above, the pose of the instrument 62 may also be displayed relative to the avatar 120. The graphic representation 90 allows the user 18 to view the pose of the instrument 62 relative to both of the image 210, 228 and/or model 78m and the avatar 120. As illustrated in FIG. 3, the image 210, 228 and/or model 78m may also be overlaid on the avatar 120.

As noted above, the avatar 120 and/or other image portions or models may be registered to the patient 14. Discussion herein of the avatar 120 and/or other image portion will be understood to refer to all possible displayed images, unless specifically stated otherwise. Registration of the avatar 120 relative to the patient 14 allows the avatar 120 to be used to assist navigation and guiding of the instrument 62, or any appropriate instrument relative to the patient 14. As the tracked position of the instrument 62 is determined with the navigation system 10, the pose of the instrument 62 may be illustrated with a graphical representation, such as the graphic representation 90 relative to the image 78 and/or the avatar 120.

The model 78m may include various portions, such as a model of the heart 15 and other portions of the patient's anatomy, such as a diaphragm 14d. It is understood, however, that various portions may be modeled or may not be modeled based upon a selected procedure. As noted above the tunneling device 62 may be used to position the sheath 67 within the subject 14. Therefore, according to various embodiments, the position of the tunneling device 62 relative to the heart 15 may be selected to be known and/or displayed on the display device 26. Therefore, the model image data 78m may include only a heart model 15m. It is understood, however, that other portions of the anatomy may also be included. As illustrated in FIGS. 14A and 14B, the model 15m may be of the heart and the pose of the instrument may be illustrated with the graphical representation 90. It is understood, however, that other image portions and/or portions may be displayed such as portions of the avatar 120, image data acquired with the imaging system 12, or other appropriate data. The discussion herein of the model of the heart 15 is merely exemplary and its illustration relative to other portions may be an example of what else may be illustrated relative to the heart model 15m.

In various embodiments, however, there may be selected to have the tunneling device 62 within a selected distance of a surface of the heart 15s. The model may include the surface 15ms represented in the model 15m. The pose of the instrument may be illustrated with the graphical representation 90. With specific reference to FIG. 14A, a distance 250 may be illustrated. According to various embodiments, the distance 250 may also be measured and displayed, such as in a distance display 254. The distance display 254 is not required. The user 18 may view the graphical representation 90 of the instrument due to the tracking of the instrument 62, 154, as discussed above. The position relative to the heart model 15m may be based upon of the image data acquired of the subject 14, as noted above, such as with the tracked ultrasound imaging system 12. Thus, the user 18 may understand the position of the tracked instrument relative to the heart 15 and the navigation system 10 may determine that the distance and pose and illustrate it as illustrated FIG. 14A. The determination of the pose of the instrument, and ultimately the lead, such as by the graphical representation 90 relative to the heart may be used for various purposes. For example, oversensing (e.g., P-wave oversensing) could be minimized as well as lead repositioning and/or re-tunneling. This may be accomplished by having clear knowledge, such as by viewing the graphical representation 90, of the relative positions of the tunnel instrument and at least a portion of the heart, such as the atrium.

Turning reference to FIG. 14B, the graphical representation 90 of the instrument may be illustrated a second or different distance 260 relative to the heart surface 15ms of the heart model 15m. The distance 260 may be displayed in the distance display 254. The user 18, therefore, may view the display 26 and understand a pose of the instrument 62, 154 relative to the heart 15 by viewing the graphical representation 90 relative to the heart model 15m. Again, as discussed above, the displayed image portion may be a model, acquired image data, images based upon the image data, or other appropriate information. Regardless, the user may view the display 26 and understand the pose of the instrument relative to the heart surface 15ms.

The tunneling instrument 62 may be selected to form a tunnel relative to the heart 15. In various embodiments, a pose of the tunneling device 62 as close as possible to the heart without puncturing or interfering with heart tissue may be selected. For example, it may be selected to position the tunneling device 62 within 2 mm of the surface of the heart 15s. Therefore, the navigation of the tunneling instrument 62 relative to the heart 15 may be displayed on the display device 26 to provide the pose information to the user 18. Thus, the user 18 may determine intraoperatively, such as and substantially real time, movement and positioning of the tunneling device 62. Due to the navigation and tracking of the tunneling device 67 and the displayed image, such as the model 15m of the heart 15, the user may view the display device 26 without requiring real time image data being acquired of the subject 14. For example, a fluoroscopic imaging system may not be present and operated to acquire the position information regarding the pose of the tunneling device 62 relative to the heart 15. Rather, the tracked pose of the tunneling device 62 may be determined and displayed on the display device 26. Therefore, the image displayed on the display device 26 may include only a model of the heart, a model of various portions of the subject 14, images based upon ultrasound image data acquired with the ultrasound imaging system 12, and/or selected fluoroscopic images. It is understood by one skilled in the art, however, that the tracked pose of the instrument 62 may be displayed only relative to the model 15 and/or other images with the display device 26.

As discussed above, with reference to FIGS. 1 through 14A and 14B, the instrument 62, 154 may be tracked relative to the subject 14 and illustrated by a graphical representation 90 on the display device 26. The instrument 62 may include a substernal tunneling device to form a tunnel relative to the heart 15 of the subject 14. Thereafter, an implant may be positioned, such as the lead 154. In various embodiments of the substernal tunneling device 62 may position the sheath 67 and thereafter the lead 154 is positioned therein. The sheath 67 may then be withdrawn to maintain the lead 154 within the subject 14.

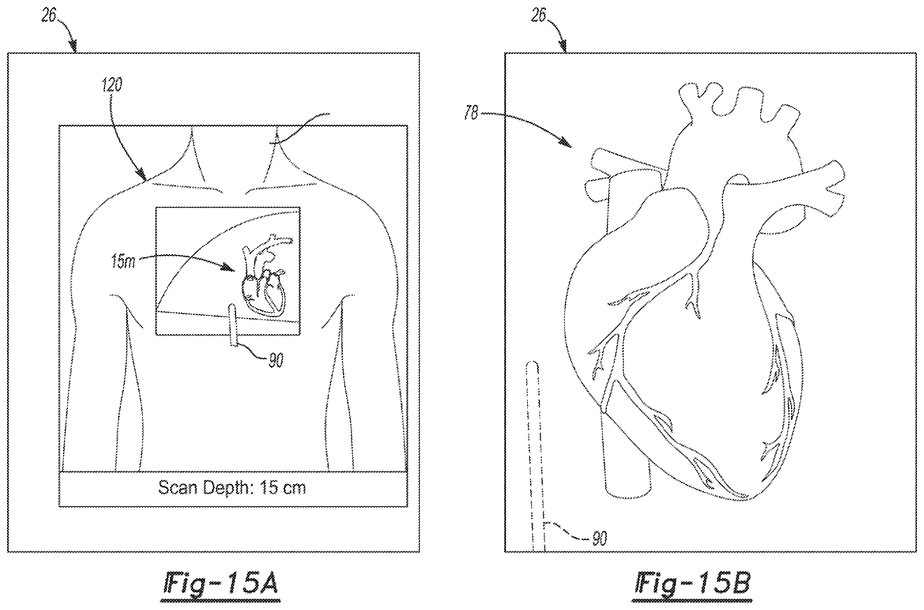

With continuing reference to FIGS. 1 through 14A and 14B, and additional reference to FIGS. 15A to 17B, the procedure for positioning the lead 154 will be discussed. In various embodiments, with initial reference to FIGS. 15A and 15B, the avatar 120 may be displayed on the display device 26. Illustrated relative thereto may also be image data and/or the model 15m of the heart 15. A graphical representation of the tunneling device 62 may be illustrated relative to the avatar 120 and/or the model 15m. The user 18 may view the graphical representation 90 and determine an appropriate or selected position of the tunneling device 62 based upon the tracked pose of the tunneling device 62 illustrated on the display 26 as the graphical representation 90. Additionally or alternatively, the display device 26 may illustrate image data 78 and/or an image based upon image data. Therefore, the image 78 may be based upon image data acquired with of the ultrasound imaging system 12. Nevertheless, the image data may also be registered to the subject 14, as discussed above. A graphical representation of the tunneling device 62 may also be displayed as the graphical representation 90 relative to the image 78. This allows the user 18 to understand the pose of the tunneling device 62 relative to the subject 14. The user 18 may view any appropriate image data, such as the avatar 120, the model 15m, or image data 78, or other appropriate representations to understand oppose of the tunneling device 62 relative to the heart 15 of the subject 14.

The tunneling device 62 may be moved relative to the subject 14 as it is tracked by the navigation system 10. The tracking of the tunneling device 62 allows for the graphical representation 90, as noted above, to be displayed as a tracked pose relative to the displayed image on the display device 26. Once the tunneling device is at a selected pose, the tunneling device may be withdrawn and the sheath 67 maintained within the subject. For example, the graphical representation 90 may illustrate that the tunneling device 62 is substantially axially positioned relative to a medial portion of the heart 16, under the sternum, or at other appropriate or selected pose. Further, the graphical representation may illustrate that the tunneling device is near the heart 15. For example, the graphical representation 90 may be used to determine or confirm that the tunneling device is within at least about 1 millimeter (mm) from the heart 15, in contact with the heart 15, within 2 mm of the heart, or other appropriate pose. In various embodiments, it may be selected to position and confirm, via the graphical representation, that the tunneling device is within 0.5 mm to about 5 mm of a surface of the heart 15. The instrument may be positioned between a sternum and the heart of the subject, subcutaneously, intercostally, intrapleurally, pericardially, epicardially, and into a posterior mediastinum, or combinations thereof.

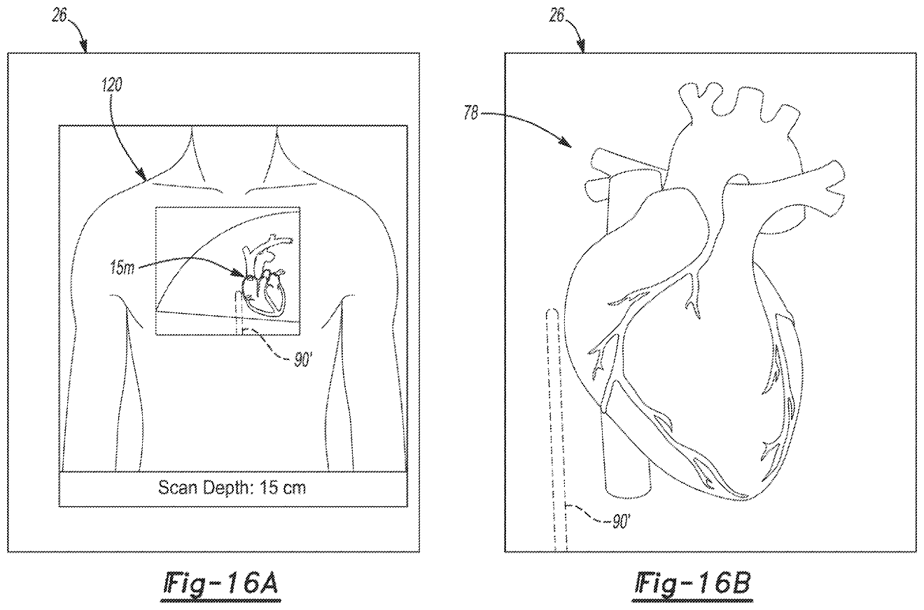

The lead 154 may then be positioned through the sheath 67 within the subject 14. A graphical representation 90′ may be displayed relative to the avatar 120 and/or the image data 78, is illustrated in FIGS. 16A and 16B. The implant 154 may be tracked with the navigation system 10, as also discussed above. In various embodiments, an EP tracking system may be used to track the lead 154 within the subject 14. For example, at least the first coil 156 and/or the ring electrode 158 may be used to sense an impedance within the subject 14. The tracked pose may then be determined and illustrated or displayed on the display device 26 as the graphical representation 90′. As the lead 154 is positioned within the subject 14, therefore, the pose may also be known. The user 18 may also, therefore, understand the pose of the lead 154 as it is positioned within the subject 14.

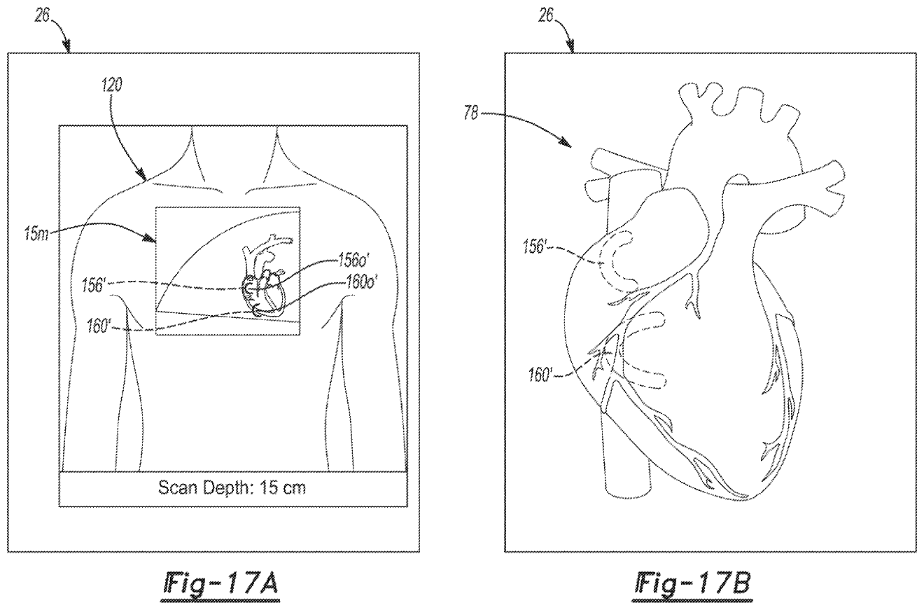

The sheath 67, as noted above, may be used to assist in positioning the lead 154. The sheath 67 may then be withdrawn once the lead is at a selected position, such as superiorly positioned within the subject 14. As the sheath is withdrawn, which may be substantially linear or straight, as illustrated in FIG. 2, the lead 154 may move to an implanted shape or orientation, as illustrated in FIGS. 17A and 17B.

The lead 154 may be selected to include a shape that is non-symmetrical relative to the central axis of a lead (e.g., has one or more “C” shapes), such as defined by the coil electrodes 156, 160. In embodiments in which the coil electrodes 156, 162 are “C”-shaped, it may be selected to have the C openings of the coil electrodes 156, 162 open or be oriented relative to the heart 15 of the subject 14 in a selected manner. As illustrated in FIGS. 17A and 17B, the pose of the coil electrodes 156, 160, or any one of the two may be illustrated with a selected graphical representation 156′ and/or 160′. It is understood, however, that the lead 154 may have or form other shapes such as “S”, “N”, “W”, or other geometric shapes, including a serpentine shape. Further, the lead 154 may include more than one shape and/or more than one occurrence of any shape. Thus, regardless of the shape of the lead, the configuration may be determined as discussed herein regarding the “C” shape, but for the selected or pre-determined selected shape.

The coil graphical representations 156′, 160′ may be displayed relative to the avatar 120, the heart model 15m, and/or the image data 78. The coil orientation may illustrate that the “C” shape has an open portion or side that may be directed or opened toward a side of the subject 14, such as the left side of the subject 14. The graphical representations 156′, 160′ may illustrate this relative to the images displayed. For example, as illustrated in FIG. 17A, an open portion 1560′ may be illustrated to open towards a left side of the avatar 120 and/or an open portion 1600′ may be illustrated to open toward the right side of the avatar 120 that represents the left side of the patient 14. Thus, the user 18 may view the graphical representations 156′, 160′ of the lead 154 to understand the orientation of the coils 156, 160 within the subject 14. The user 18 may also then determine whether the oil openings are open in a selected or desired orientation and either reposition and/or complete the implantation of the lead 154.

The selected pose of the lead 154 within the subject 14 may assist in achieving various factors. For example, the closer the lead 154, including the electrodes 156, 160 thereof, is to the heart 15 the lower the impulse (e.g., voltage or amperage) necessary to achieve a therapy of the heart 15. For example, a defibrillation and/or pacing impulse may be lower the closer the electrodes 156, 160 are to the selected portion of the heart 15. In other words, less power may be necessary to achieve a selected defibrillation result if the lead 154 is 1 mm from the heart 15 as opposed to 5 mm from the heart 15. At least by the tracking of the instrument 62 and/or the lead 154, the pose of the lead may be determined and/or confirmed within the subject 14.

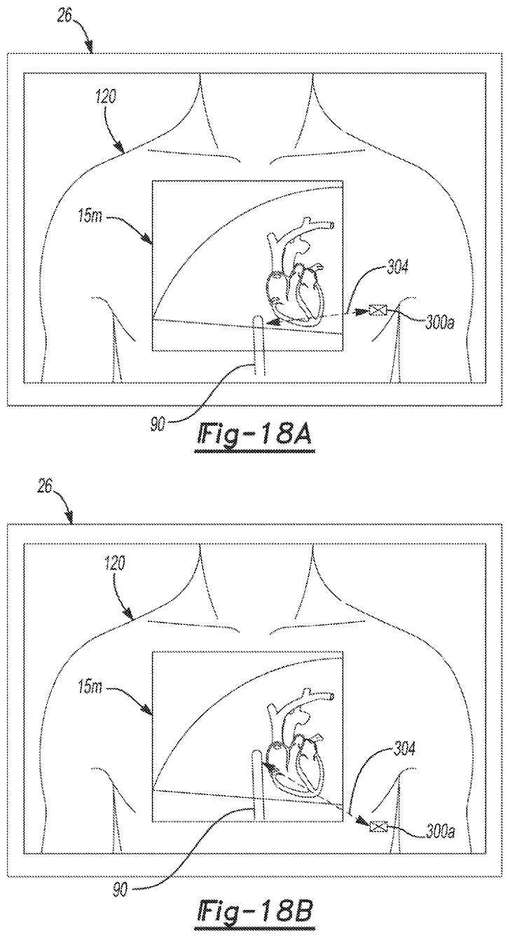

Further, a final and/or precise known placement of the lead 154 and/or the electrodes 156, 160 may assist in related or similar outcomes. In various embodiments, an implant 300, such as an implantable cardiac device (ICD) and the lead 154. Further, a vector 304 between the ICD 300 and the lead 154 may be determined based on at least the known pose within the subject 14. The known pose may be based at least on the tracking of the lead 154 and/or the ICD 300. Further, the poses may be illustrated, as illustrated in FIG. 18 alone and/lor with a representation of the vector 304.

In other words, briefly, the ICD 300 pose can be identified, at least within a known range, with the tracking sensor(s). A volume of the heart within the vector 304 may also be known based on the image data, modeling, and/or morphing of the atlas heart. Also the tunneling path and/or final pose of the lead may be known with the tracking. This pose and/or volume information may allow modeling of the defibrillation vector 304 and may be displayed on the user interface 26 for assessment. Both tunnel and device (e.g., ICD 300) location can be adjusted if needed. With data regarding final electrode position, shape, and orientation, a more accurate final prediction could be made about defibrillation vector and estimated defibrillation threshold could be displayed to the user 18.

With reference to FIGS. 18A and 18B, the navigation system 10 may be used in determining a placement and/or for planning a placement of the lead 154. As discussed above, the tunneling instrument 62 may be tracked with the instrument tracking device. This allows a pose of the instrument 62 to be displayed with the graphical representation 90. Also, a planned path may be illustrated as extending from an end, such as a distal end of the tunneling portion 62c. Thus, the graphical representation 90 may illustrate a real time pose of the instrument 62 and/or a planned path or placement of the instrument 62.

In addition, the heart 15 may also be illustrated with the display 26. As discussed above, the heart 15 may be imaged and images may be displayed. Further, a model of the heart 15m may be rendered and displayed. The model 15m may be based on the current patient or subject 14, such as being morphed based on image data of the heart 15. Thus, the volume of the heart may be determined, such as by morphing a volumetric model with the image data of the subject 14. The image of the heart may also be reconstructed using a selected portion, such as all, of the image data collected regarding the heart of the subject. This may be similar as to how computed tomography images are reconstructed. Thus, with ultrasound image data multiple ultrasound sweeps may be stitched together to reconstruct an image of the heart, such as a partial or full image of the heart of the subject.

The ICD 300 and/or a representation thereof may also be tracked. The ICD 300 may be tracked relative to the subject before or during a procedure. The tracking of the ICD 300 may, therefore, allow its pose to be determined and displayed with the display device 26.

With initial reference to FIG. 18A, the user 18 may position the tracked instrument, such as the tunneling instrument 62 relative to the subject 14. A graphical representation, such as the graphical representation 90 of the instrument may be displayed on the display device 26, such as relative to the avatar 120 and/or the heart model 15m. The graphical representation illustrates the pose of the instrument 62, 154 that is tracked with the tracking system and the graphical representation 90 may be positioned on the display device 26 relative to the avatar 120 or the image based upon its tracked pose. The graphical representation may also be based on a tracked lead and/or be understood to represent an implanted location of a lead once the instrument is removed.

In addition, the user 18 may position a tracked instrument, a tracked ICD 300, or other tracked portion relative to the subject 14 away from the tunneling instrument 62 to determine a second of ICDS position. Additionally or alternatively, the user may select a position on the image that may be selected for placement of the ICD 300. Regardless of the method (e.g., tracking an instrument or ICD and/or manual selection), a graphical representation 300a may be illustrated on the display device 26 to illustrate a or selected (e.g., planned) pose of the ICD 300 or tracked portion to illustrate a current or planned pose of the ICD 300. The tracked pose of the ICD 300 may be determined based on positioning the ICD 300 in the subject with the instrument 90 and/or a selected tracked instrument. Thus, the graphical representation 90 of the instrument and the graphical representation 300a of the ICD 300 may be illustrated substantially simultaneously on the display device 26.

The display may also include the representation of the heart 15, such as the model 15m. The graphical representations 90, 300a may be illustrated relative to the heart model 15m. This may allow the user 18 to view and understand the pose of the heart 15 relative to a planned or current pose of the ICD 300 and/or the instrument 62 or the lead 154.

In addition, a selected processor may determine and generate a graphical representation of a line or vector 304. The line 304 may be illustrated as a substantially straight line between the tip of the instrument graphical representation 90 and the ICD graphical representation 300a, as illustrated in FIG. 18A. The processor 74 may execute instructions to determine the line between points, such as predetermined points in the graphical representations 90 and 300a.

The user 18 may view the line or vector 304 between the ICD representation 300a and the instrument graphical representation 90 and determine or select whether the vector 304 is selected for the subject 14. The tracked pose of the instrument 62 may be used in a planning step, such as prior to tunneling within the subject 14, and illustrated on the display 26. Similarly, the tracked portion to represent the ICD 300 may be tracked in a planning step prior to implantation in the subject 14. Therefore, the display on the display device 26, as illustrated in FIG. 18A, may display a planning and/or confirmation of a procedure to position the lead 154 and the ICD 300 in the subject 14. The user 18 may confirm of a desired pose, is illustrated in FIG. 18A, or reposition or illustrates different poses.

With reference to FIG. 18B, for example, the graphical representation 90 may be in a different pose relative to the displayed portions of the subject, such as the model 15m and the avatar 120. Similarly, the ICD representation 300a may be at a different pose relative to the illustrated portions of the subject. Similarly, the vector 304 may also be different, therefore, based upon the alternative or second pose of the graphical representation 90 of the instrument and the graphical representation of the ICD 300a. Thus, the user 18 may view at least two poses of the instrument 62, 154 and/or the ICD 300 with the display device to plan and/or confirm a selected pose or position for both the lead and the ICD 300.

By allowing the user 18 to view the tracked poses with the graphical representations 90, 300a, the user 18 may plan or select the poses or positions for the implanted portions including the lead 154 and the ICD 300. As discussed herein, the graphical representation 90 of the tool may related to a final position of one or more leads as the lead may be positioned at the tip or along or near a position of the tool represented by the graphical representation 90. Thus, an electrical connection between a lead (generally represented by the graphical representation) and the ICD 300 represented by the representation 300a may be estimated. The user 18 before, may attempt to optimize, such as attempting to provide a minimal energy to cardiovert the heart 15. For example, the user may select to attempt to include a defibrillation threshold (DFT) that is minimal (i.e., as low as possible to achieve cardioversion of the heart) in the subject 14. By having the distance, represented by the vector 304, minimized or crossing a selected portion of the heart 15, the user 18 may assess and/or attempt to minimize the DFT. The analysis by the user 18 may be complemented by the model 15m which includes a representation of the heart 15 of the subject 14. As discussed above, the model 15 may be based upon an atlas and/or various images of the heart 15 of the subject 14. Therefore, the user may view and/or understand a volume of the heart 15 of the particular subject 14 based upon the model 15m. The model 15m may be morphed to the patient heart 15 that is the current patient based upon the image data acquired with of the imaging system 12 used to morphed and/or form the model 15m.

This allows the user 18 to assist in positioning the ICD 300 and lead 154 in the subject in a selected position, which may be an optimal or preplanned position. The position of the ICD 300 and the lead 154 may be used to optimize or reduce a DFT and/or other considerations. For example, positioning the lead 154 at a selected pose relative to the heart, such as not past in an atrial position, may assist in reducing or minimizing over sensing of a P-wave of the heart. Graphical representation 90 and/or the graphical representation of the ICD 300a may allow the user 18 to confirm and/or plan for placement of the implants in the subject 14.

The graphical representation may further assist in minimizing pacing capture threshold such as by distance to heart tissue, avoiding pericardial/epicardial fat pads. Also, an identification of location of the tool 67c may incorporate sensors, such as impedance or pressure sensors to assist in identifying the type of tissue near the tip of the insertion tool. The determination of a position of the implant may also minimize pain and/or a sensation. This may incorporate other sensor data into the image. When tissue response to a stimulation pulse indicates pain/sensation is likely, overlay with image.

The graphical representation 304 may be illustrated in any appropriate manner. A line or vector may be selected. Alternatively or additionally, however, other shapes and/or representations may be made. The graphical representation may illustrate an energy, such as a defibrillation energy and/or pacing energy that travels in a volume of space. The exact or determined direction and/or volumetric path is a factor of many parameters or features. Key parameters or features include the location of the lead, orientation of the lead, ICD position, and heart position. Additional parameters or features factors may include body fat, body thickness, etc. may affect the volumetric path, such as of the defibrillation pulse. These parameters or features affect the electrical resistance and/or impedance of the path as well as the boundary conditions.

Thus, the graphical representation 304 may include at least one of a line, a vector, at least one of a straight line between the first graphical representation and the second graphical representation, a 2D or 3D shading, a line from an average location of one or more electrodes between near the first graphical representation and the second graphical representation, a 2D or 3D projection on to the heart representing a cardiac tissue that would exceed a threshold energy, a line or multidimensional display that includes a calculated estimate of a selected parameter including at least one of defibrillation threshold, pacing threshold, and/or at least one of a pacing threshold, ventricular sensing amplitude, or atrial sensing amplitude. Any representation may also include a calculated estimate of a clinically relevant parameter (e.g., defibrillation threshold, pacing threshold, etc.).

Further, any of the information may be used to assist in placement of any one of the lead and/or the ICD. The graphical representation may be used to select and/or move the lead, the ICD, change an orientation, or the like. Data from the instrument tracking data and the patient anatomy may be used to calculate and/or visualize an optimal location for a device location based on predicted clinically relevant parameters (e.g., defibrillation threshold, pacing threshold, ventricular sensing amplitude, atrial sensing amplitude, etc.). Data from an ideal device location and patient anatomy may also be used to optimize a lead placement. A system and/or method may include determining an optimal placement of at least one of the lead or the ICD based on the third graphical representation.

The image 78, as discussed above, may be based upon image data that is generated with the imaging system 12 that may be the ultrasound imaging system, as discussed above. Therefore, the image 78 may be based upon image data that is acquired with nonionizing image data, such as with a fluoroscope or other x-ray imaging systems. Thus, the image that is displayed on the display device 26 may be displayed based upon image data acquired of the subject 14 without exposing the subject 14 to ionizing radiation. In addition, the imaging system 12 may be a small imaging system that may be easily and quickly moved relative to the subject 14 for efficient and time-saving image data acquisition of the subject 14.

In addition, as noted above, the image 78 may be based only on image data and/or may be based upon a model generated with the image data. Further, a model, such as the model 15m, discussed above, may be based upon morphing an atlas or other general images based upon the acquired image data. Regardless of whether the image is based directly on image data and/or based upon more fitting a predetermined or prior generated image to current image data, the display may display a three-dimensional image and/or may be based upon a three-dimensional image. Thus, the image, as discussed above, may be a two-dimensional image based upon image data acquired with the imaging system 12 or may be a three-dimensional image that is based upon image data acquired with the imaging system 12 alone and/or in combination with other data. Regardless the display device 26 may display representations of the subject 14 and/or portions of the subject, such as the heart 15, is based only on nonionizing imaging systems. Further the image may be a two-dimensional image or three-dimensional volumetric image to represent a volume of the subject 14, including a volume of the heart 15.

The various information that may be used to evaluate various features, such as placement of leads and/or the ICD 300 may further include other generally collected information. For example, non-visual data may include point measurements of sensing amplitude (e.g., R-wave, P-wave), pacing capture threshold, subject sensation risk (e.g., pacing pulse), pressure, impedance, etc., may be either interpolated graphically or used for ‘mapping’ the implant location for different success criteria.

Further, as discussed above various portions, such as the instrument including the introducer and/or the lead, may be tracked. Various tracking systems are discussed above, such as EM tracking systems and/or impedance tracking systems. It is understood that any appropriate tracking system may be used. Impedance is one method of tracking the lead and electrode position, but various embodiments may include electromagnetic tracking or fiber bragg grating optical tracking could be used. For example, a stylet may contain the EM tracking coil or optical fiber. This stylet would then be used in a stylet lumen of the lead. This would enable positional and orientation tracking of the lead during implant. The stylet could then be removed once the lead is placed.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit, a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

### Conclusion

Other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-18B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system to track a device relative to a subject, comprising:
a display device configured to display an image that includes a representation of one or more internal portions of the subject;
an instrument configured to be moved relative to the heart of the subject, wherein the instrument includes a distal portion configured to be percutaneously positioned in the subject and a handle configured to move the distal portion;
a tracking system;
a tracking device configured to be tracked by the tracking system, wherein the tracking device is associated with the instrument;
a processing unit configured to execute instructions to:
generate the image that includes the representation of the one or more internal portions of the subject;
register the image to the subject; and
generate a graphical representation of a pose of the instrument relative to the generated image; and
a display device configured to display both the image and the graphical representation;
wherein the display is operable to illustrate at least a distance of the instrument relative to the heart of the subject based at least on the displayed image and the graphical representation.

2. The system of claim 1, further comprising:
an imaging system configured to generate image data of the one or more internal portions of the subject;
wherein processing unit is configured to generate the image with the image data.

3. The system of claim 2, further comprising:
an imaging system tracker, wherein the imaging system tracker is configured to be tracked by the tracking system to determine a pose of the imaging system relative to the subject;
wherein the imaging system is an ultrasound imaging system configured to generate the image data of the subject in a plane relative to an ultrasound transducer.

4. The system of claim 3, wherein the generated image is an image based on the ultrasound image data;
wherein the generated image is registered to the subject based at least on the imaging system tracker pose.

5. The system of claim 2, wherein the processing unit is configured to execute further instructions to:
recall a heart atlas;
morph the heart atlas based on the image data of the subject to generate a morphed atlas; and
display the morphed atlas as the image and the generated graphical representation relative to the morphed atlas.

6. The system of claim 5, wherein the atlas is registered to a generated image of the heart at least by an identification of a landmark in both the atlas and the generated image of the heart.

7. The system of claim 1, further comprising:
an implant configured to be implanted within the subject near the heart, wherein the implant is configured to include an electrode configured to deliver energy to the heart of the subject;
an implant tracking device associated with the implant;
wherein the implant tracking device is configured to be tracked with the tracking system to determine a pose of the implant.

8. The system of claim 7, wherein the tracking system includes an electrical impedance tracking system;
wherein the implant tracking device is the electrode of the implant;
wherein the processing unit is configured to execute further instructions to determine at least an orientation of the electrode.

9. The system of claim 1, wherein the instrument is configured to be at least one of positioned between a sternum and the heart of the subject, subcutaneously, intercostally, intrapleurally, pericardially, epicardially, into a posterior mediastinum, or combinations thereof.

10. A method to track a device relative to a heart of a subject, comprising:
providing an instrument including (1) a distal portion configured to be percutaneously positioned and moved relative to the subject and (2) a handle configured to be manipulated to move the distal portion;
tracking a tracking device associated with the provided instrument;
operating a processing unit to execute instructions to:
generate an image to represent at least a portion of the heart of the subject;
register the image to the subject;
determine a pose of the provided instrument based on the tracking of the tracking device; and
generate a graphical representation of the instrument relative to the generated image based on the determined pose of the provided instrument; and
displaying with a display device both the generated image and the graphical representation;
wherein the display is operable to illustrate at least a distance of the instrument relative to the heart of the subject based at least on the displayed image and the graphical representation.

11. The method of claim 10, further comprising:
acquiring image data of the subject with an imaging system;
operating the processing unit to generate the image with the acquired image data.
tracking the imaging system with an imaging system tracker to determine a pose of the imaging system relative to the subject;
providing the imaging system as an ultrasound imaging system;
wherein operating the processing unit to register the image to the subject is based at least on the imaging system determined pose.

12. The method of claim 10, wherein operating the processing unit further includes:
recalling a heart atlas;
morphing the heart atlas based on the image data of the subject to generate a morphed atlas; and
displaying the morphed atlas as the image and the generated graphical representation relative to the morphed atlas.

13. The method of claim 10, further comprising:
providing an implant configured to be implanted within the subject near the heart,
providing the implant to include an electrode to deliver energy to the heart of the subject;
tracking the implant with an implant tracking device associated with the implant; and
determining a pose of the implant based on tracking the implant.

14. The method of claim 13, further comprising:
determining an orientation of the electrode including at least a determination of an orientation of non-symmetrical lead elements relative to a central axis of the implant.

15. A system to track a device relative to a heart of a subject, comprising:
a display device configured to display an image that includes a representation of the subject;
an instrument configured to be moved relative to the heart of the subject, wherein the instrument includes (1) a distal portion configured to be positioned transcutaneous between a sternum and the heart of the subject and (2) a handle configured to move the distal portion;
a lead configured to be positioned within the subject;
a tracking system;
an instrument tracking device configured to be tracked by the tracking system, wherein the instrument tracking device is associated with the instrument and tracked by the tracking system to determine an instrument pose;
a lead tracking portion configured to be tracked by the tracking system, wherein the lead tracking device is associated with the lead and tracked by the tracking system to determine a lead pose;
a processing unit configured to execute instructions to:
generate the image that represents at least a portion of the heart of the subject based at least on a determined pose of the subject;
register the image to the subject; and
generate a graphical representation of the determined pose of at least one of the instrument or the lead relative to the generated image; and
a display device configured to display both the image and the graphical representation to illustrate the determined pose in real time relative to the heart of the subject, wherein the real time display illustrates at least a distance of the instrument relative to the heart of the subject based at least on the displayed image and the graphical representation.
